(19) United States
(12) Patent Application Publication
(10) Pub. No.: US 2002/0155589 A1
(43) Pub. Date: Oct. 24, 2002

(54) Biochemical analysis unit and biochemical analyzing method using the same

(75) Inventor: Tohru Tsuchiya, Kanagawa (JP)

Correspondence Address: SUGHRUE MION, PLLC, 2100 Pennsylvania Avenue, NW, Washington, DC, 20037-3213, US

(73) Assignee: FUJI PHOTO FILM CO., LTD.

(21) Appl. No.: 10/112,080

(22) Filed: Apr. 1, 2002

(30) Foreign Application Priority Data
Mar. 30, 2001 (JP) 2001-101028

(51) Int. Cl. C12M 1/34; C12Q 1/68; G01N 33/53; G01N 33/542

(52) U.S. Cl. 435/287.2; 435/6; 435/7.9

(57) ABSTRACT

A biochemical analysis unit includes a plurality of absorptive regions formed spaced apart from each other by covering a surface of an absorptive substrate made of an absorptive material with a material capable of attenuating radiation energy and/or light energy. According to this biochemical analysis unit, it is possible to prevent noise caused by the scattering of electron beams released from a radioactive labeling substance from being generated in biochemical analysis data even in the case of forming spots of specific binding substances on the surface of a carrier at high density, specifically binding the spot-like specific binding substance with a substance derived from a living organism and labeled with a radioactive substance to selectively label the spot-like specific binding substances with a radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

### BACKGROUND OF THE INVENTION

The present invention relates to a biochemical analysis unit and a biochemical analyzing method using the same and, particularly, to a biochemical analysis unit and a biochemical analyzing method which can prevent noise caused by the scattering of electron beams released from a radioactive labeling substance from being generated in biochemical analysis data even in the case of forming spots of specific binding substances on the surface of a carrier at a high density, which can specifically bind with a substance derived from a living organism and whose sequence, base length, composition and the like are known, specifically binding the spot-like specific binding substances with a substance derived from a living organism labeled with a radioactive substance to selectively label the spot-like specific binding substances with the radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism; and can prevent noise caused by the scattering of chemiluminescent emission and/or fluorescence emission released from a labeling substance which generates chemiluminescent emission when it contacts a chemiluminescent substrate and/or a fluorescent substance from being generated in biochemical analysis data even in the case of forming spots of specific binding substances on the surface of a carrier at high density, which can specifically bind with a substance derived from a living organism and whose sequence, base length, composition and the like are known, specifically binding the spot-like specific binding substance with a substance derived from a living organism labeled with, in addition to a radioactive labeling substance or instead of a radioactive labeling substance, a labeling substance which generates chemiluminescent emission when it contacts a chemiluminescent substrate and/or a fluorescent substance to selectively label the spot-like specific binding substances therewith, thereby obtaining a biochemical analysis unit, photoelectrically detecting chemiluminescent emission and/or fluorescence emission released from the biochemical analysis unit to produce biochemical analysis data, and analyzing the substance derived from a living organism.

### DESCRIPTION OF THE PRIOR ART

An autoradiographic analyzing system using as a detecting material for detecting radiation a stimulable phosphor which can absorb, store and record the energy of radiation when it is irradiated with radiation and which, when it is then stimulated by an electromagnetic wave having a specified wavelength, can release stimulated emission whose light amount corresponds to the amount of radiation with which it was irradiated is known, which comprises the steps of introducing a radioactively labeled substance into an organism, using the organism or a part of the tissue of the organism as a specimen, superposing the specimen and a stimulable phosphor sheet formed with a stimulable phosphor layer for a certain period of time, storing and recording radiation energy in a stimulable phosphor contained in the stimulable phosphor layer, scanning the stimulable phosphor layer with an electromagnetic wave to excite the stimulable phosphor, photoelectrically detecting the stimulated emission released from the stimulable phosphor to produce digital image signals, effecting image processing on the obtained digital image signals, and reproducing an image on displaying means such as a CRT or the like or a photographic film (see, for example, Japanese Patent Publication No. 1-60784, Japanese Patent Publication No. 1-60782, Japanese Patent Publication No. 4-3952 and the like).

There is further known chemiluminescence analysis system comprising the steps of employing, as a detecting material for light, a stimulable phosphor which can absorb and store the energy of light upon being irradiated therewith and release a stimulated emission whose amount is proportional to that of the received light upon being stimulated with an electromagnetic wave having a specific wavelength range, selectively labeling a fixed high molecular substance such as a protein or a nucleic acid sequence with a labeling substance which generates chemiluminescent emission when it contacts a chemiluminescent substance, contacting the high molecular substance selectively labeled with the labeling substance and the chemiluminescent substance, storing and recording the chemiluminescent emission in the wavelength of visible light generated by the contact of the chemiluminescent substance and the labeling substance in the stimulable phosphor contained in a stimulable phosphor layer formed on a stimulable phosphor sheet, scanning the stimulable phosphor layer with an electromagnetic wave to excite the stimulable phosphor, photoelectrically detecting the stimulated emission released from the stimulable phosphor to produce digital signals, effecting data processing on the obtained digital signals, and reproducing data on displaying means such as a CRT or a recording material such as a photographic film (see for example, U.S. Pat. No. 5,028,793, UK Patant Application 2,246,197 A and the like).

Unlike the system using a photographic film, according to these systems using the stimulable phosphor as a detecting material, development, which is chemical processing, becomes unnecessary. Further, it is possible reproduce a desired image by effecting image processing on the obtained image data and effect quantitative analysis using a computer. Use of a stimulable phosphor in these processes is therefore advantageous.

On the other hand, a fluorescence analyzing system using a fluorescent substance as a labeling substance instead of a radioactive labeling substance in the autoradiographic analyzing system is known. According to this system, it is possible to study a genetic sequence, study the expression level of a gene, and to effect separation or identification of protein or estimation of the molecular weight or properties of protein or the like. For example, this system can perform a process including the steps of distributing a plurality of DNA fragments on a gel support by means of electrophoresis after a fluorescent dye was added to a solution containing a plurality of DNA fragments to be distributed, or distributing a plurality of DNA fragments on a gel support containing a fluorescent dye, or dipping a gel support on which a plurality of DNA fragments have been distributed by means of electrophoresis in a solution containing a fluorescent dye, thereby labeling the electrophoresed DNA fragments, exciting the fluorescent dye by a stimulating ray to cause it to release fluorescent light, detecting the released fluorescent light to produce an image and detecting the distribution of the DNA fragments on the gel support. This system can also perform a process including the steps of distributing a plurality of DNA fragments on a gel support by means of electrophoresis, denaturing the DNA fragments, transferring at least a part of the denatured DNA fragments onto a transfer support such as a nitrocellulose support by the Southern-blotting method, hybridizing a probe prepared by labeling target DNA and DNA or RNA complementary thereto with the denatured DNA fragments, thereby selectively labeling only the DNA fragments complementary to the probe DNA or probe RNA, exciting the fluorescent dye by a stimulating ray to cause it to release fluorescent light, detecting the released fluorescent light to produce an image and detecting the distribution of the target DNA on the transfer support. This system can further perform a process including the steps of preparing a DNA probe complementary to DNA containing a target gene labeled by a labeling substance, hybridizing it with DNA on a transfer support, combining an enzyme with the complementary DNA labeled by a labeling substance, causing the enzyme to contact a fluorescent substance, transforming the fluorescent substance to a fluorescent substance having fluorescent light releasing property, exciting the thus produced fluorescent substance by a stimulating ray to release fluorescent light, detecting the fluorescent light to produce an image and detecting the distribution of the target DNA on the transfer support. This fluorescence detecting system is advantageous in that a genetic sequence or the like can be easily detected without using a radioactive substance.

Similarly, there is known a chemiluminescence analysis system comprising the steps of fixing a substance derived from a living organism such as a protein or a nucleic acid sequence on a support, selectively labeling the substance derived from a living organism with a labeling substance which generates chemiluminescent emission when it contacts a chemiluminescent substrate, contacting the substance derived from a living organism and selectively labeled with the labeling substance and the chemiluminescent substrate, photoelectrically detecting the chemiluminescent emission in the wavelength of visible light generated by the contact of the chemiluminescent substrate and the labeling substance to produce digital image signals, effecting image processing thereon, and reproducing a chemiluminescent image on a display means such as a CRT or a recording material such as a photographic film, thereby obtaining information relating to the high molecular substance such as genetic information.

Further, a micro-array analyzing system has been recently developed, which comprises the steps of using a spotting device to drop at different positions on the surface of a carrier such as a slide glass plate, a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substances using a hybridization method or the like with a substance derived from a living organism such as a cell, virus, hormone, tumor marker, enzyme, antibody, antigen, abzyme, other protein, a nuclear acid, cDNA, DNA or mRNA, which is gathered from a living organism by extraction, isolation or the like or is further subjected to chemical processing, chemical modification or the like and which is labeled with a labeling substance such as a fluorescent substance, dye or the like, thereby forming a micro-array, irradiating the micro-array with a stimulating ray, photoelectrically detecting light such as fluorescence emission released from a labeling substance such as a fluorescent substance, dye or the like, and analyzing the substance derived from a living organism. This micro-array analyzing system is advantageous in that a substance derived from a living organism can be analyzed in a short time period by forming a number of spots of specific binding substances at different positions of the surface of a carrier such as a slide glass plate at high density and hybridizing them with a substance derived from a living organism and labeled with a labeling substance.

In addition, a macro-array analyzing system using a radioactive labeling substance as a labeling substance has been further developed, which comprises the steps of using a spotting device to drop at different positions on the surface of a carrier such as a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substance using a hybridization method or the like with a substance derived from a living organism such as a cell, virus, hormone, tumor marker, enzyme, antibody, antigen, abzyme, other protein, a nuclear acid, cDNA, DNA or mRNA, which is gathered from a living organism by extraction, isolation or the like or is further subjected to chemical processing, chemical modification or the like and which is labeled with a radioactive labeling substance, thereby forming a macroarray, superposing the macro-array and a stimulable phosphor sheet formed with a stimulable phosphor layer, exposing the stimulable phosphor layer to a radioactive labeling substance, irradiating the stimilable phosphor layer with a stimulating ray to excite the stimulable phosphor, photoelectrically detecting the stimulated emission released from the stimulable phosphor to produce biochemical analysis data, and analyzing the substance derived from a living organism.

However, in the macro-array analyzing system using a radioactive labeling substance as a labeling substance, when the stimulable phosphor layer is exposed to a radioactive labeling substance, since radiation energy of the radioactive labeling substance contained in spots formed on the surface of a carrier such as a membrane filter is very large, electron beams released from the radioactive labeling substance contained in the individual spots are scattered in the carrier such as a membrane filter, thereby impinging on regions of the stimulable phosphor layer that should be exposed to the radioactive labeling substance contained in neighboring spots, or electron beams released from the radioactive labeling substance contained in the individual spots are scattered and mixed with the electron beams released from the radioactive labeling substance contained in neighboring spots and then impinge on regions of the stimulable phosphor layer to generate noise in biochemical analysis data produced by photoelectrically detecting stimulated emission and to lower the accuracy of biochemical analysis when a substance derived from a living organism is analyzed by quantifying the radiation amount of each spot. The accuracy of biochemical analysis is markedly degraded when spots are formed closely to each other at high density.

In order to solve these problems by preventing noise caused by the scattering of electron beams released from radioactive labeling substance contained in neighboring spots, it is inevitably required to increase the distance between neighboring spots and this makes the density of the spots lower and the test efficiency lower.

Further, in the field of biochemical analysis, it is often required to analyze a substance derived from a living organism by forming a plurality of spot-like regions containing specific binding substances spot-like formed at different positions on the surface of a carrier such as a membrane filter or the like, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, specifically binding, using a hybridization method or the like, the specific binding substances contained in the plurality of spot-like regions with a substance derived from a living organism labeled with, in addition to a radioactive labeling substance, a labeling substance which generates chemiluminescent emission when it contacts a chemiluminescent substrate and/or a fluorescent substance, thereby selectively labeling the plurality of spot-like regions and after exposing a stimulable phosphor layer to the radioactive labeling substance or prior to exposing a stimulable phosphor layer to the radioactive labeling substance, causing it to contact a chemiluminescent substrate, thereby photoelectrically detecting the chemiluminescent emission in the wavelength of visible light, and/or irradiating it with a stimulating ray, thereby photoelectrically detecting fluorescence emission released from a fluorescent substance. In these cases, chemiluminescent emission or fluorescence emission released from the plurality of spot-like regions is scattered in the carrier such as a membrane filter or chemiluminescent emission or fluorescence emission released from any particular spot-like region is scattered and mixed with chemiluminescent emission or fluorescence emission released from neighboring spot-like regions, thereby generating noise in biochemical analysis data produced by photoelectrically detecting chemiluminescent emission and/or fluorescence emission.

Furthermore, in the field of biochemical analysis, it is often required to analyze a substance derived from a living organism by forming a plurality of spot-like regions containing specific binding substances at different positions on the surface of a carrier such as a membrane filter or the like, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, specifically binding, using a hybridization method or the like, the specific binding substances contained in the plurality of spot-like regions with a substance derived from a living organism labeled with a labeling substance which generates chemiluminescent emission when it contacts a chemiluminescent substrate, thereby selectively labeling the plurality of spot-like regions, causing the plurality of spot-like regions to come into contact with a chemiluminescent substrate, exposing a stimulable phosphor layer to chemiluminescent emission in the wavelength of visible light generated by the contact of the chemiluminescent substance and the labeling substance, thereby storing the energy of chemiluminescent emission in the stimulable phosphor layer, irradiating the stimulable phosphor layer with a stimulating ray, and photoelectrically detecting stimulated emission released from the stimulable phosphor layer, thereby effecting biochemical analysis. In this case, chemiluminescent emission released from any particular spot-like region is scattered in the carrier such as a membrane filter and mixed with chemiluminescent emission released from neighboring spot-like regions, thereby generating noise in biochemical analysis data produced by photoelectrically detecting chemiluminescent emission.

### SUMMARY OF THE INVENTION

It is therefore an object of the present invention to provide a biochemical analysis unit which can prevent noise caused by the scattering of electron beams released from a radioactive labeling substance from being generated in biochemical analysis data even in the case of forming spots of specific binding substances on the surface of a carrier at high density, which can specifically bind with a substance derived from a living organism and whose sequence, base length, composition and the like are known, specifically binding the spot-like specific binding substance with a substance derived from a living organism and labeled with a radioactive substance to selectively label the spot-like specific binding substances with a radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

It is another object of the present invention to provide a biochemical analysis unit which can prevent noise caused by the scattering of chemiluminescent emission and/or fluorescence emission released from a labeling substance which generates chemiluminescent emission when it contacts a chemiluminescent substrate and/or a fluorescent substance from being generated in biochemical analysis data even in the case of forming spots of specific binding substances on the surface of a carrier at high density, which can specifically bind with a substance derived from a living organism and whose sequence, base length, composition and the like are known, specifically binding the spot-like specific binding substance with a substance derived from a living organism and labeled with, in addition to a radioactive labeling substance or instead of a radioactive labeling substance, a labeling substance which generates chemiluminescent emission when it contacts a chemiluminescent substrate and/or a fluorescent substance to selectively label the spot-like specific binding substances therewith, thereby obtaining a biochemical analysis unit, photoelectrically detecting chemiluminescent emission and/or fluorescence emission released from the biochemical analysis unit to produce biochemical analysis data, and analyzing the substance derived from a living organism.

It is a further object of the present invention to provide a biochemical analyzing method which can effect quantitative biochemical analysis with high accuracy by producing biochemical analysis data based on a biochemical analysis unit obtained by forming spots of specific binding substances on the surface of a carrier at high density, which can specifically bind with a substance derived from a living organism and whose sequence, base length, composition and the like are known, specifically binding the spot-like specific binding substances with a substance derived from a living organism and labeled with a radioactive labeling substance, a labeling substance which generates chemiluminescent emission when it contacts a chemiluminescent substrate and/or a fluorescent substance, thereby selectively labeling the spot-like specific binding substances therewith.

The above other objects of the present invention can be accomplished by a biochemical analysis unit comprising a plurality of absorptive regions formed spaced apart from each other by covering a surface of an absorptive substrate made of an absorptive material with a material capable of attenuating radiation energy and/or light energy.

In one mode of use of the biochemical analysis unit according to this aspect of the present invention, the plurality of absorptive regions are formed spaced apart from each other by covering the surface of the absorptive substrate with a material capable of attenuating radiation energy, specific binding substances, which can specifically bind with a substance derived from a living organism and whose sequence, base length, composition and the like are known, are spotted in the plurality of absorption regions formed in the biochemical analysis unit at a high density and a substance derived from a living organism and labeled with a radioactive substance is specifically bound, using a hybridization method or the like, with the specific binding substances contained in the plurality of absorptive regions, thereby selectively labeling the plurality of absorptive regions therewith. The biochemical analysis unit is then disposed so as to face a stimulable phosphor layer, thereby exposing the stimulable phosphor layer to the radioactive labeling substance contained in the plurality of absorptive regions. At this time, since electron beams (β rays) released from the radioactive labeling substance contained in the individual absorptive regions are attenuated by the covering of the material capable of attenuating radiation energy formed on the surface of the absorptive substrate around the individual absorptive regions, the stimulable phosphor layer can be exposed to only electron beams (β rays) released though the surfaces of the absorptive regions, thereby enabling only regions of the stimulable phosphor layer facing the individual absorptive regions to be selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy.

In another mode of use of the biochemical analysis unit according to this aspect of the present invention, the plurality of absorptive regions are formed spaced apart from each other by covering the surface of the absorptive substrate with a material capable of attenuating light energy, specific binding substances, which can specifically bind with a substance derived from a living organism and whose sequence, base length, composition and the like are known, are spotted in the plurality of absorption regions formed in a biochemical analysis unit at a high density and a substance derived from a living organism and labeled with a labeling substance which generates chemiluminescent emission when it contacts a chemiluminescent substrate and/or a fluorescent substance, instead of with a radioactive labeling substance, is specifically bound, using a hybridization method or the like, with the specific binding substances contained in the plurality of absorptive regions, thereby selectively labeling the plurality of absorptive regions therewith. Biochemical analysis data are then produced by photoelectrically detecting chemiluminescent emission generated by the contact of a chemiluminescent substrate and the labeling substance in response to contact of the biochemical analysis unit and the chemiluminescent substrate and/or fluorescence emission released from the fluorescent substance in response to irradiating the biochemical analysis unit with a stimulating ray. At this time, since chemiluminescent emission and/or fluorescence emission is attenuated by the covering of the material capable of attenuating light energy formed on the surface of the absorptive substrate around the individual absorptive regions, detection of only chemiluminescent emission and/or fluorescence emission released though the surfaces of the absorptive regions is possible. Therefore, it is possible to efficiently prevent noise caused by the scattering and mixing of chemiluminescent emission released from the labeling substance which generates chemiluminescent emission when it contacts a chemiluminescent substrate and/or fluorescence emission released from the fluorescent substance contained absorptive regions next to each other from being generated in biochemical analysis data produced by photoelectrically detecting chemiluminescent emission and/or fluorescence emission.

In a further mode of use of the biochemical analysis unit according to this aspect of the present invention, the plurality of absorptive regions are formed spaced apart from each other by covering the surface of the absorptive substrate with a material capable of attenuating radiation energy and light energy, specific binding substances, which can specifically bind with a substance derived from a living organism and whose sequence, base length, composition and the like are known, are spotted in the plurality of absorption regions formed in a biochemical analysis unit at a high density and a substance derived from a living organism and labeled with a labeling substance which generates chemiluminescent emission when it contacts a chemiluminescent substrate and/or a fluorescent substance, in addition to a radioactive labeling substance, is specifically bound, using a hybridization method or the like, with the specific binding substances contained in the plurality of absorptive regions, thereby selectively labeling the plurality of absorptive regions therewith. In the case where the biochemical analysis unit is then disposed so as to face a stimulable phosphor layer, thereby exposing the stimulable phosphor layer to the radioactive labeling substance contained in the plurality of absorptive regions, since electron beams (β rays) released from the radioactive labeling substance contained in the individual absorptive regions are attenuated by the covering of the material capable of attenuating radiation energy and light energy formed on the surface of the absorptive substrate around the individual absorptive regions, the stimulable phosphor layer can be exposed to only electron beams (β rays) released though the surfaces of the absorptive regions, thereby enabling only regions of the stimulable phosphor layer facing the individual absorptive regions to be selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy. On the other hand, when biochemical analysis data are produced by photoelectrically detecting chemiluminescent emission generated by the contact of a chemiluminescent substrate and the labeling substance in response to contact of the biochemical analysis unit and the chemiluminescent substrate and/or fluorescence emission released from the fluorescent substance in response to irradiating the biochemical analysis unit with a stimulating ray, since chemiluminescent emission and/or fluorescence emission is attenuated by the covering of the material capable of attenuating radiation energy and light energy formed on the surface of the absorptive substrate around the individual absorptive regions, detecion of only chemiluminescent emission and/or fluorescence emission released though the surfaces of the absorptive regions is possible. Therefore, it is possible to efficiently prevent noise caused by the scattering and mixing of chemiluminescent emission released from the labeling substance which generates chemiluminescent emission when it contacts a chemiluminescent substrate and/or fluorescence emission released from the fluorescent substance contained absorptive regions next to each other from being generated in biochemical analysis data produced by photoelectrically detecting chemiluminescent emission and/or fluorescence emission.

The above and other objects of the present invention can also be accomplished by a biochemical analysis unit comprising a plurality of absorptive regions formed spaced apart from each other by covering a surface of an absorptive substrate made of an absorptive material with a material capable of attenuating radiation energy and/or light energy, the plurality of absorptive regions being selectively labeled with at least one kind of labeling substance selected from a group consisting of a radioactive labeling substance, a labeling substance which generates chemiluminescent emission when it contacts a chemiluminescent substrate and a fluorescent substance by spotting specific binding substances whose sequence, base length, composition and the like are known therein and specifically binding a substance derived from a living organism and labeled with at least one kind of said labeling substance with the specific binding substances.

In the present invention, the case where a plurality of absorptive regions are selectively labeled with a fluorescent substance as termed herein includes the case where a plurality of absorptive regions are selectively labeled with a fluorescent substance by selectively binding a substance derived from a living organism and labeled with a fluorescent substance with specific binding substances contained in the plurality of absorptive regions and the case where a plurality of absorptive regions are selectively labeled with a fluorescent substance by selectively binding a substance derived from a living organism and labeled with a hapten, binding an antibody for the hapten labeled with an enzyme which generates a fluorescent substance when it contacts a fluorescent substrate with the hapten by an antigen-antibody reaction, and causing the enzyme bound with the hapten to come into contact with the fluorescent substrate to generate a fluorescent substance.

Further, in the present invention, the case where a plurality of absorptive regions are selectively labeled with a labeling substance which generates chemiluminescent emission when it contacts a chemiluminescent substrate as termed herein includes the case where a plurality of absorptive regions are selectively labeled with a labeling substance which generates chemiluminescent emission when it contacts a chemiluminescent substrate by selectively binding a substance derived from a living organism and labeled with a labeling substance which generates chemiluminescent emission when it contacts a chemiluminescent substrate and the case where a plurality of absorptive regions are selectively labeled with a labeling substance which generates chemiluminescent emission when it contacts a chemiluminescent substrate by selectively binding a substance derived from a living organism and labeled with a hapten, and binding an antibody for the hapten labeled with an enzyme which generates chemiluminescent emission when it contacts a chemiluminescent substrate with the hapten by an antigen-antibody reaction.

According to this aspect of the present invention, in the case where the plurality of absorptive regions are formed spaced apart from each other by covering the surface of the absorptive substrate with a material capable of attenuating radiation energy, when the biochemical analysis unit is disposed so as to face a stimulable phosphor layer, thereby exposing the stimulable phosphor layer to the radioactive labeling substance contained in the plurality of absorptive regions, since electron beams (β rays) released from the radioactive labeling substance contained in the individual absorptive regions are attenuated by the covering of the material capable of attenuating radiation energy formed on the surface of the absorptive substrate around the individual absorptive regions, the stimulable phosphor layer can be exposed to only electron beams (β rays) released though the surfaces of the absorptive regions, thereby enabling only regions of the stimulable phosphor layer facing the individual absorptive regions to be selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy.

Further, according to this aspect of the present invention, in the case where the plurality of absorptive regions are formed spaced apart from each other by covering the surface of the absorptive substrate with a material capable of attenuating light energy, when biochemical analysis data are produced by photoelectrically detecting chemiluminescent emission generated by the contact of a chemiluminescent substrate and the labeling substance in response to contact of the biochemical analysis unit and the chemiluminescent substrate and/or fluorescence emission released from the fluorescent substance in response to irradiating the biochemical analysis unit with a stimulating ray, since chemiluminescent emission and/or fluorescence emission is attenuated by the covering of the material capable of attenuating light energy formed on the surface of the absorptive substrate around the individual absorptive regions, detection of only chemiluminescent emission and/or fluorescence emission released though the surfaces of the absorptive regions is possible. Therefore, it is possible to efficiently prevent noise caused by the scattering and mixing of chemiluminescent emission released from the labeling substance which generates chemiluminescent emission when it contacts a chemiluminescent substrate and/or fluorescence emission released from the fluorescent substance contained absorptive regions next to each other from being generated in biochemical analysis data produced by photoelectrically detecting chemiluminescent emission and/or fluorescence emission.

Furthermore, according to this aspect of the present invention, in the case where the plurality of absorptive regions are formed spaced apart from each other by covering the surface of the absorptive substrate with a material capable of attenuating radiation energy and light energy, when the biochemical analysis unit is disposed so as to face a stimulable phosphor layer, thereby exposing the stimulable phosphor layer to the radioactive labeling substance contained in the plurality of absorptive regions, since electron beams (β rays) released from the radioactive labeling substance contained in the individual absorptive regions are attenuated by the covering of the material capable of attenuating radiation energy and light energy formed on the surface of the absorptive substrate around the individual absorptive regions, the stimulable phosphor layer can be exposed to only electron beams (β rays) released though the surfaces of the absorptive regions, thereby enabling only regions of the stimulable phosphor layer facing the individual absorptive regions to be selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy. On the other hand, when biochemical analysis data are produced by photoelectrically detecting chemiluminescent emission generated by the contact of a chemiluminescent substrate and the labeling substance in response to contact of the biochemical analysis unit and the chemiluminescent substrate and/or fluorescence emission released from the fluorescent substance in response to irradiating the biochemical analysis unit with a -stimulating ray, since chemiluminescent emission and/or fluorescence emission is attenuated by the covering of the material capable of attenuating light energy formed on the surface of the absorptive substrate around the individual absorptive regions, detection of only chemiluminescent emission and/or fluorescence emission released though the surfaces of the absorptive regions is possible. Therefore, it is possible to efficiently prevent noise caused by the scattering and mixing of chemiluminescent emission released from the labeling substance which generates chemiluminescent emission when it contacts a chemiluminescent substrate and/or fluorescence emission released from the fluorescent substance contained absorptive regions next to each other from being generated in biochemical analysis data produced by photoelectrically detecting chemiluminescent emission and/or fluorescence emission.

In the present invention, illustrative examples of the combination of hapten and antibody include digoxigenin and antidigoxigenin antibody, theophylline and anti-theophylline antibody, fluorosein and anti-fluorosein antibody, and the like. Further, the combination of biotin and avidin, antigen and antibody may be utilized instead of the combination of hapten and antibody.

The above and other objects of the present invention can be also accomplished by a biochemical analyzing method comprising the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating radiation energy and specifically binding a substance derived from a living organism and labeled with the radioactive labeling substance with the specific binding substances, thereby selectively labeling the plurality of absorptive regions with the radioactive labeling substance, superposing the biochemical analysis unit on a stimulable phosphor sheet on which a stimulable phosphor layer is formed, thereby exposing the stimulable phosphor layer to the radioactive labeling substance selectively contained in the plurality of absorptive regions, irradiating the stimulable phosphor layer exposed to the radioactive labeling substance with a stimulating ray to excite the stimulable phosphor layer, photoelectrically detecting stimulated emission released from the stimulable phosphor layer to produce biochemical analysis data, and effecting biochemical analysis based on the thus produced biochemical analysis data.

According to this aspect of the present invention, when the biochemical analysis unit is superposed on the stimulable phosphor sheet on which a stimulable phosphor layer is formed, thereby exposing the stimulable phosphor layer to the radioactive labeling substance selectively contained in the plurality of absorptive regions, since electron beams (β rays) released from the radioactive labeling substance contained in the individual absorptive regions are attenuated by the covering of the material capable of attenuating radiation energy formed on the surface of the absorptive substrate around the individual absorptive regions, the stimulable phosphor layer can be exposed to only electron beams (β rays) released though the surfaces of the absorptive regions, thereby enabling only regions of the stimulable phosphor layer facing the individual absorptive regions to be selectively exposed to electron beams (β rays). Therefore, it is possible to efficiently prevent noise caused by the exposure of regions of the stimulable phosphor layer facing the individual absorptive regions to electron beams (β rays) released from an absorptive region next thereto from being generated in biochemical analysis data and to produce biochemical analysis data having high quantitative accuracy.

In a preferred aspect of the present invention, the stimulable phosphor layer of the stimulable phosphor sheet includes a plurality of stimulable phosphor regions formed by charging stimulable phosphor into a plurality of holes formed in a support made of a material capable of attenuating radiation energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to the radioactive labeling substance selectively contained in the plurality of absorptive regions.

According to this preferred aspect of the present invention, since the stimulable phosphor layer of the stimulable phosphor sheet includes a plurality of stimulable phosphor regions formed by charging stimulable phosphor into a plurality of holes formed in a support made of a material capable of attenuating radiation energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to the radioactive labeling substance selectively contained in the plurality of absorptive regions, electron beams (β rays) released from the radioactive labeling substance selectively contained in the individual absorptive regions can be prevented from being scattered in the corresponding stimulable phosphor layer region and reaching a stimulable phosphor layer region facing an absorptive region next thereto and it is therefore possible to reliably expose each of the stimulable phosphor layer regions formed on the stimulable phosphor sheet to only the radioactive labeling substance contained in the corresponding absorptive region and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by selectively labeling the plurality of absorptive regions with a fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively labeling the plurality of absorptive regions with a fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data, fluorescence emission released from the fluorescent substance is attenuated by the covering of the material capable of attenuating radiation energy and light energy formed on the surface of the absorptive substrate around the individual absorptive regions, thereby enabling only fluorescence emission released through the surfaces of the plurality of absorptive regions to be photoelectrically detected, and it is therefore possible to efficiently prevent noise caused by the scattering and mixing of fluorescence emission released from the fluorescent substances contained in the absorptive regions next to each other in biochemical analysis data produced by photoelectrically detecting fluorescence emission and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by specifically binding the substance derived from a living organism and labeled with a fluorescent substance in addition to the radioactive labeling substance with the specific binding substance contained in the plurality of absorptive regions, thereby selectively labeling the plurality of absorptive regions, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by specifically binding the substance derived from a living organism and labeled with a fluorescent substance in addition to the radioactive labeling substance with the specific binding substance contained in the plurality of absorptive regions, thereby selectively labeling the plurality of absorptive regions, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data, fluorescence emission released from the fluorescent substance is attenuated by the covering of the material capable of attenuating radiation energy and light energy formed on the surface of the absorptive substrate around the individual absorptive regions, thereby enabling only fluorescence emission released through the surfaces of the plurality of absorptive regions to be photoelectrically detected, and it is therefore possible to efficiently prevent noise caused by the scattering and mixing of fluorescence emission released from the fluorescent substances contained in the absorptive regions next to each other in biochemical analysis data produced by photoelectrically detecting fluorescence emission and to produce biochemical analysis data having high quantitative accuracy.

In another preferred aspect of the present invention, the material capable of attenuating radiation energy has a further capability to attenuate light energy and the biochemical analyzing method further comprises the steps of preparing the biochemical analysis unit by selectively binding the substance derived from a living organism and labeled with a hapten in addition to the radioactive labeling substance with the specific binding substance contained in the plurality of absorptive regions, binding an antibody for the hapten labeled with an enzyme having a property to produce a fluorescent substance when it contacts a fluorescence substrate with the hapten by an antigen-antibody reaction and contacting the enzyme bound with the hapten and the fluorescence substrate to produce a fluorescent substance, thereby selectively labeling the plurality of absorptive regions with the fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively binding the substance derived from a living organism and labeled with a hapten with the specific binding substance contained in the plurality of absorptive regions, binding an antibody for the hapten labeled with an enzyme having a property to produce a fluorescent substance when it contacts a fluorescence substrate by an antigen-antibody reaction and contacting the enzyme bound with the hapten and the fluorescence substrate to produce a fluorescent substance, thereby selectively labeling the plurality of absorptive regions with the fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data, fluorescence emission released from the fluorescent substance is attenuated by the covering of the material capable of attenuating radiation energy and light energy formed on the surface of the absorptive substrate around the individual absorptive regions, thereby enabling only fluorescence emission released through the surfaces of the plurality of absorptive regions to be photoelectrically detected, and it is therefore possible to efficiently prevent noise caused by the scattering and mixing of fluorescence emission released from the fluorescent substances contained in the absorptive regions next to each other in biochemical analysis data produced by photoelectrically detecting fluorescence emission and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data, chemiluminescent emission generated by the contact of the chemiluminescent substrate and the labeling substance is attenuated by the covering of the material capable of attenuating radiation energy and light energy formed on the surface of the absorptive substrate around the individual absorptive regions, thereby enabling only chemiluminescence emission released through the surfaces of the plurality of absorptive regions to be photoelectrically detected, and it is therefore possible to efficiently prevent noise caused by the scattering and mixing of chemiluminescence emission released from the labeling substances contained in the absorptive regions next to each other in biochemical analysis data produced by photoelectrically detecting chemiluminescence emission and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, detecting chemiluminescent emission released from the labeling substance to record biochemical analysis data in a recording material, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, detecting chemiluminescent emission released from the labeling substance to record biochemical analysis data in a recording material, and effecting biochemical analysis based on the thus produced biochemical analysis data, chemiluminescent emission generated by the contact of the chemiluminescent substrate and the labeling substance is attenuated by the covering of the material capable of attenuating radiation energy and light energy formed on the surface of the absorptive substrate around the individual absorptive regions, thereby enabling only chemiluminescence emission released through the surfaces of the plurality of absorptive regions to be detected to record biochemical analysis data in a recording material such as a photographic film, and it is therefore possible to efficiently prevent noise caused by the scattering and mixing of chemiluminescence emission released from the labeling substances contained in the absorptive regions next to each other in biochemical analysis data recorded in the recording material such as a photographic film by detecting chemiluminescence emission and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by specifically binding the substance derived from a living organism and labeled with a labeling substance which generates chemiluminescent emission when it contacts a chemiluminescent substrate in addition to the radioactive labeling substance with the specific binding substance contained in the plurality of absorptive regions, thereby selectively labeling the plurality of absorptive regions, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by specifically binding the substance derived from a living organism and labeled with a labeling substance which generates chemiluminescent emission when it contacts a chemiluminescent substrate in addition to the radioactive labeling substance with the specific binding substance contained in the plurality of absorptive regions, thereby selectively labeling the plurality of absorptive regions, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data, chemiluminescent emission generated by the contact of the chemiluminescent substrate and the labeling substance is attenuated by the covering of the material capable of attenuating radiation energy and light energy formed on the surface of the absorptive substrate around the individual absorptive regions, thereby enabling only chemiluminescent emission released through the surfaces of the plurality of absorptive regions to be photoelectrically detected, and it is therefore possible to efficiently prevent noise caused by the scattering and mixing of chemiluminescent emission released from the labeling substances contained in the absorptive regions next to each other in biochemical analysis data produced by photoelectrically detecting chemiluminescent emission and to produce biochemical analysis data having high quantitative accuracy.

In a further preferred aspect of the present invention, the material capable of attenuating radiation energy has a further capability to attenuate light energy and the biochemical analyzing method further comprises the steps of preparing the biochemical analysis unit by specifically binding the substance derived from a living organism and labeled with a labeling substance which generates chemiluminescent emission when it contacts a chemiluminescent substrate in addition to the radioactive labeling substance with the specific binding substance contained in the plurality of absorptive regions, thereby selectively labeling the plurality of absorptive regions, causing the biochemical analysis unit to come into contact with the chemiluminescent substrate, detecting chemiluminescent emission released from the labeling substance to record biochemical analysis data in a recording material, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by specifically binding the substance derived from a living organism and labeled with a labeling substance which generates chemiluminescent emission when it contacts a chemiluminescent substrate in addition to the radioactive labeling substance with the specific binding substance contained in the plurality of absorptive regions, thereby selectively labeling the plurality of absorptive regions, causing the biochemical analysis unit to come into contact with the chemiluminescent substrate, detecting chemiluminescent emission released from the labeling substance to record biochemical analysis data in a recording material, and effecting biochemical analysis based on the thus produced biochemical analysis data, chemiluminescent emission generated by the contact of the chemiluminescent substrate and the labeling substance is attenuated by the covering of the material capable of attenuating radiation energy and light energy formed on the surface of the absorptive substrate around the individual absorptive regions, thereby enabling only chemiluminescent emission released through the surfaces of the plurality of absorptive regions to be detected to record biochemical analysis data in a recording material such as a photographic film, and it is therefore possible to efficiently prevent noise caused by the scattering and mixing of chemiluminescent emission released from the labeling substances contained in the absorptive regions next to each other in biochemical analysis data recorded in the recording material such as a photographic film by detecting chemiluminescent emission and to produce biochemical analysis data having high quantitative accuracy.

In another preferred aspect of the present invention, the material capable of attenuating radiation energy has a further capability to attenuate light energy and the biochemical analyzing method further comprises the steps of preparing the biochemical analysis unit by selectively binding the substance derived from a living organism and labeled with a hapten in addition to the radioactive labeling substance with the specific binding substance contained in the plurality of absorptive regions, binding an antibody for the hapten labeled with an enzyme having a property to generate chemiluminescent emission when it contacts a chemiluminescent substrate by an antigen-antibody reaction, causing the biochemical analysis unit to come into contact with the chemiluminescent substance, thereby generating chemiluminescent emission, photoelectrically detecting the chemiluminescent emission released from the enzyme to produce biochemical analysis data, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively binding the substance derived from a living organism and labeled with a hapten with the specific binding substance contained in the plurality of absorptive regions, binding an antibody for the hapten labeled with an enzyme having a property to generate chemiluminescent emission when it contacts a chemiluminescent substrate by an antigen-antibody reaction, causing the biochemical analysis unit to come into contact with the chemiluminescent substance, thereby generating chemiluminescent emission, photoelectrically detecting the chemiluminescent emission released from the enzyme to produce biochemical analysis data, and effecting biochemical analysis based on the thus produced biochemical analysis data, chemiluminescent emission generated by the contact of the chemiluminescent substrate and the enzyme is attenuated by the covering of the material capable of attenuating radiation energy and light energy formed on the surface of the absorptive substrate around the individual absorptive regions, thereby enabling only chemiluminescent emission released through the surfaces of the plurality of absorptive regions to be photoelectrically detected, and it is therefore possible to efficiently prevent noise caused by the scattering and mixing of chemiluminescent emission released from the enzyme contained in the absorptive regions next to each other in biochemical analysis data produced by photoelectrically detecting chemiluminescent emission and to produce biochemical analysis data having high quantitative accuracy.

In another preferred aspect of the present invention, the material capable of attenuating radiation energy has a further capability to attenuate light energy and the biochemical analyzing method further comprises the steps of preparing the biochemical analysis unit by selectively binding the substance derived from a living organism and labeled with a hapten in addition to the radioactive labeling substance with the specific binding substance contained in the plurality of absorptive regions, binding an antibody for the hapten labeled with an enzyme having a property to generate chemiluminescent emission when it contacts a chemiluminescent substrate by an antigen-antibody reaction, causing the biochemical analysis unit to come into contact with the chemiluminescent substance, thereby generating chemiluminescent emission, detecting the chemiluminescent emission released from the enzyme to record biochemical analysis data in a recording material, and effecting biochemical analysis based on the thus produced biochemical analysis data.

According to this preferred aspect of the present invention, since the material capable of attenuating radiation energy has a further capability to attenuate light energy and the biochemical analyzing method further comprises the steps of preparing the biochemical analysis unit by selectively binding the substance derived from a living organism and labeled with a hapten with the specific binding substance contained in the plurality of absorptive regions, binding an antibody for the hapten labeled with an enzyme having a property to generate chemiluminescent emission when it contacts a chemiluminescent substrate by an antigen-antibody reaction, causing the biochemical analysis unit to come into contact with the chemiluminescent substance, thereby generating chemiluminescent emission, detecting the chemiluminescent emission released from the enzyme to record biochemical analysis data in a recording material, and effecting biochemical analysis based on the thus produced biochemical analysis data, chemiluminescent emission generated by the contact of the chemiluminescent substrate and the enzyme is attenuated by the covering of the material capable of attenuating radiation energy and light energy formed on the surface of the absorptive substrate around the individual absorptive regions, thereby enabling only chemiluminescent emission released through the surfaces of the plurality of absorptive regions to be detected to record biochemical analysis data in a recording material such as a photographic film, and it is therefore possible to efficiently prevent noise caused by the scattering and mixing of chemiluminescent emission released from the enzyme contained in the absorptive regions next to each other in biochemical analysis data recorded in the recording material such as a photographic film by detecting chemiluminescent emission and to produce biochemical analysis data having high quantitative accuracy.

The above and other objects of the present invention can be also accomplished by a biochemical analyzing method comprising the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and selectively labeling the plurality of absorptive regions with a fluorescent substance, irradiating the biochemical analysis unit with a stimulating ray to stimulate the fluorescent substance, photoelectrically detecting fluorescence emission released from the fluorescent substance to produce biochemical analysis data, and effecting biochemical analysis based on the thus produced biochemical analysis data.

According to this aspect of the present invention, since the biochemical analyzing method comprises the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating radiation energy and selectively labeling the plurality of absorptive regions with a fluorescent substance, irradiating the biochemical analysis unit with a stimulating ray to stimulate the fluorescent substance, photoelectrically detecting fluorescence emission released from the fluorescent substance to produce biochemical analysis data, and effecting biochemical analysis based on the thus produced biochemical analysis data, fluorescence emission released from the fluorescent substance is attenuated by the covering of the material capable of attenuating light energy formed on the surface of the absorptive substrate around the individual absorptive regions, thereby enabling only fluorescence emission released through the surfaces of the plurality of absorptive regions to be photoelectrically detected, and it is therefore possible to efficiently prevent noise caused by the scattering and mixing of fluorescence emission released from the fluorescent substances contained in the absorptive regions next to each other in biochemical analysis data produced by photoelectrically detecting fluorescence emission and to produce biochemical analysis data having high quantitative accuracy.

In a preferred aspect of the present invention, the biochemical analysis unit is prepared by specifically binding a substance derived from a living organism and labeled with a fluorescent substance with the specific binding substances contained in the plurality of absorptive regions, thereby selectively labeling the plurality of absorptive regions.

In another preferred aspect of the present invention, the biochemical analysis unit is prepared by selectively binding a substance derived from a living organism and labeled with hapten with the specific binding substances contained in the plurality of absorptive regions, binding an antibody for the hapten labeled with an enzyme having a property to produce a fluorescent substance when it contacts a fluorescence substrate with the hapten by an antigen-antibody reaction and contacting the enzyme bound with the hapten and the fluorescence substrate to produce a fluorescent substance, thereby selectively labeling the plurality of absorptive regions with the fluorescent substance.

The above and other objects of the present invention can be also accomplished by a biochemical analyzing method comprising the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and selectively labeling the plurality of absorptive regions with a substance derived from a living organism and labeled with a labeling substance which generates chemiluminescent emission when it contacts a chemiluminescent substrate, contacting the biochemical analysis unit and the chemiluminescent substrate, detecting chemiluminescent emission released from the labeling substance to produce biochemical analysis data, and effecting biochemical analysis based on the thus produced biochemical analysis data.

According to this aspect of the present invention, since the biochemical analyzing method comprises the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and selectively labeling the plurality of absorptive regions with a substance derived from a living organism and labeled with a labeling substance which generates chemiluminescent emission when it contacts a chemiluminescent substrate, contacting the biochemical analysis unit and the chemiluminescent substrate, detecting chemiluminescent emission released from the labeling substance to produce biochemical analysis data, and effecting biochemical analysis based on the thus produced biochemical analysis data, chemiluminescent emission generated by the contact of the chemiluminescent substrate and the enzyme is attenuated by the covering of the material capable of attenuating light energy formed on the surface of the absorptive substrate around the individual absorptive regions, thereby enabling only chemiluminescent emission released through the surfaces of the plurality of absorptive regions to be detected, and it is therefore possible to efficiently prevent noise caused by the scattering and mixing of chemiluminescent emission released from the enzyme contained in the absorptive regions next to each other in biochemical analysis data produced by detecting chemiluminescent emission and to produce biochemical analysis data having high quantitative accuracy.

In a preferred aspect of the present invention, the biochemical analysis data are produced by photoelectrically detecting chemiluminescent emission released from the labeling substance.

In another preferred aspect of the present invention, the biochemical analysis data are produced and recorded in a recording material by detecting chemiluminescent emission released from the labeling substance.

In a preferred aspect of the present invention, the biochemical analysis unit is prepared by specifically binding a substance derived from a living organism and labeled with a labeling substance which generates chemiluminescent emission when it contacts a chemiluminescent substrate with the specific binding substances contained in the plurality of absorptive regions, thereby selectively labeling the plurality of absorptive regions.

In another preferred aspect of the present invention, the biochemical analysis unit is prepared by selectively binding a substance derived from a living organism and labeled with hapten with the specific binding substances contained in the plurality of absorptive regions, binding an antibody for the hapten labeled with an enzyme having a property to generate chemiluminescent emission when it contacts a chemiluminescent substrate with the hapten by an antigen-antibody reaction, thereby selectively labeling the plurality of absorptive regions with the enzyme.

In a preferred aspect of the present invention, the substance derived from a living organism is specifically bound with specific binding substances by a reaction selected from a group consisting of hybridization, antigen-antibody reaction and receptor-ligand reaction.

In a preferred aspect of the present invention, the biochemical analysis unit is formed with a gripping portion by which the biochemical analysis unit can be gripped.

According to this preferred aspect of the present invention, since the biochemical analysis unit is formed with a gripping portion by which the biochemical analysis unit can be gripped, the biochemical analysis unit can be very easily handled when specific binding substances are spotted, during hybridization, during antigen-antibody reaction or during exposure operation.

The above and other objects of the present invention can be also accomplished by a biochemical analyzing method comprising the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and specifically binding a substance derived from a living organism and labeled with a labeling substance which generates chemiluminescent emission when it contacts a chemiluminescent substrate with the specific binding substances, thereby selectively labeling the plurality of absorptive regions with the labeling substance which generates chemiluminescent emission when it contacts a chemiluminescent substrate, causing the plurality of absorptive regions of the biochemical analysis unit to come into contact with the chemiluminescent substrate, thereby causing the plurality of absorptive regions to release chemiluminescent emission, superposing the biochemical analysis unit whose plurality of the absorptive regions are releasing chemiluminescent emission and a stimulable phosphor sheet formed with a stimulable phosphor layer, exposing the stimulable phosphor layer to chemiluminescent emission released from the plurality of absorptive regions of the biochemical analysis unit, thereby storing an energy of chemiluminescent emission in the stimulable phosphor layer of the stimulable phosphor sheet, irradiating the plurality of absorptive regions of the biochemical analysis unit with a stimulating ray, thereby exciting stimulable phosphor contained in the stimulable phosphor layer, photoelectrically detecting stimulated emission released from the stimulable phosphor layer of the stimulable phosphor sheet to produce biochemical analysis data, and effecting biochemical analysis based on the thus produced biochemical analysis data.

According to the present invention, since a biochemical analyzing method comprises the steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and specifically binding a substance derived from a living organism and labeled with a labeling substance which generates chemiluminescent emission when it contacts a chemiluminescent substrate with the specific binding substances, thereby selectively labeling the plurality of absorptive regions with the labeling substance which generates chemiluminescent emission when it contacts a chemiluminescent substrate, causing the plurality of absorptive regions of the biochemical analysis unit to come into contact with the chemiluminescent substrate, thereby causing the plurality of absorptive regions to release chemiluminescent emission, superposing the biochemical analysis unit whose plurality of the absorptive regions are releasing chemiluminescent emission and a stimulable phosphor sheet formed with a stimulable phosphor layer, exposing the stimulable phosphor layer to chemiluminescent emission released from the plurality of absorptive regions of the biochemical analysis unit, thereby storing an energy of chemiluminescent emission in the stimulable phosphor layer of the stimulable phosphor sheet, irradiating the plurality of absorptive regions of the biochemical analysis unit with a stimulating ray, thereby exciting stimulable phosphor contained in the stimulable phosphor layer, photoelectrically detecting stimulated emission released from the stimulable phosphor layer of the stimulable phosphor sheet to produce biochemical analysis data, and effecting biochemical analysis based on the thus produced biochemical analysis data, when the stimulable phosphor sheet is superposed on the biochemical analysis unit to expose the stimulable phosphor layer of the stimulable phosphor sheet to chemiluminescent emission released from the plurality of absorptive regions of the biochemical analysis unit, chemiluminescent emission released from the individual absorptive regions is attenuated by the covering of the material capable of attenuating light energy formed on the surface of the absorptive substrate around the individual absorptive regions and, therefore, the stimulable phosphor layer can be exposed to only chemiluminescent emission released though the surfaces of the absorptive regions, thereby enabling only regions of the stimulable phosphor layer facing the individual absorptive regions to store the energy of chemiluminescent emission. Therefore, it is possible to efficiently prevent noise caused by the scattering of chemiluminescent emission from being generated in biochemical analysis data produced by irradiating the stimulable phosphor layer storing the energy of chemiluminescent emission with a stimulating ray and photoelectrically detecting stimulated emission released from the stimulable phosphor layer and to produce biochemical analysis data having high quantitative accuracy.

In a preferred aspect of the present invention, the stimulable phosphor layer of the stimulable phosphor sheet includes a plurality of stimulable phosphor regions formed by charging stimulable phosphor into a plurality of holes formed in a support made of a material capable of attenuating light energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to chemiluminescent emission released from the plurality of absorptive regions of the biochemical analysis unit.

According to this preferred aspect of the present invention, since the stimulable phosphor layer of the stimulable phosphor sheet includes a plurality of stimulable phosphor regions formed by charging stimulable phosphor into a plurality of holes formed in a support made of a material capable of attenuating light energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to chemiluminescent emission released from the plurality of absorptive regions of the biochemical analysis unit, chemiluminescent emission released from the individual absorptive regions can be more effectively prevented from being scattered in the corresponding stimulable phosphor layer region and reaching a stimulable phosphor layer region facing an absorptive region next thereto and it is therefore possible to effectively expose each of the stimulable phosphor layer regions formed on the stimulable phosphor sheet to only chemiluminescent emission released from the corresponding absorptive region, to store the energy of chemiluminescent emission therein and to produce biochemical analysis data having high quantitative accuracy.

In a preferred aspect of the present invention, the biochemical analysis unit is prepared by specifically binding a substance derived from a living organism and labeled with a labeling substance which generates chemiluminescent emission when it contacts a chemiluminescent substrate with the specific binding substances contained in the plurality of absorptive regions, thereby selectively labeling the plurality of absorptive regions.

In another preferred aspect of the present invention, the biochemical analysis unit is prepared by selectively binding a substance derived from a living organism and labeled with hapten with the specific binding substances contained in the plurality of absorptive regions, binding an antibody for the hapten labeled with an enzyme having a property to generate chemiluminescent emission when it contacts a chemiluminescent substrate with the hapten by an antigen-antibody reaction, thereby selectively labeling the plurality of absorptive regions with the enzyme.

In a preferred aspect of the present invention, the substance derived from a living organism is specifically bound with specific binding substances by a reaction selected from a group consisting of hybridization, antigen-antibody reaction and receptor-ligand reaction.

In a preferred aspect of the present invention, the material capable of attenuating radiation energy has a property of reducing the energy of radiation to ⅕ or less when the radiation travels in the material by a distance equal to that between neighboring absorptive regions.

In a further preferred aspect of the present invention, a material capable of attenuating radiation energy has a property of reducing the energy of radiation to 1/10 or less when the radiation travels in the material by a distance equal to that between neighboring absorptive regions.

In a further preferred aspect of the present invention, a material capable of attenuating radiation energy has a property of reducing the energy of radiation to 1/50 or less when the radiation travels in the material by a distance equal to that between neighboring absorptive regions.

In a further preferred aspect of the present invention, a material capable of attenuating radiation energy has a property of reducing the energy of radiation to 1/100 or less when the radiation travels in the material by a distance equal to that between neighboring absorptive regions.

In a further preferred aspect of the present invention, a material capable of attenuating radiation energy has property of reducing the energy of radiation to 1/500 or less when the radiation travels in the material by a distance equal to that between neighboring absorptive regions.

In a further preferred aspect of the present invention, a material capable of attenuating radiation energy has a property of reducing the energy of radiation to 1/1000 or less when the radiation travels in the material by a distance equal to that between neighboring absorptive regions.

In a preferred aspect of the present invention, a material capable of attenuating light energy has a property of reducing the energy of light to ⅕ or less when the light travels in the material by a distance equal to that between neighboring absorptive regions.

In a further preferred aspect of the present invention, a material capable of attenuating light energy has a property of reducing the energy of light to 1/10 or less when the light travels in the material by a distance equal to that between neighboring absorptive regions.

In a further preferred aspect of the present invention, a material capable of attenuating light energy has a property of reducing the energy of light to 1/50 or less when the light travels in the material by a distance equal to that between neighboring absorptive regions.

In a further preferred aspect of the present invention, a material capable of attenuating light energy has a property of reducing the energy of light to 1/100 or less when the light travels in the material by a distance equal to that between neighboring absorptive regions.

In a further preferred aspect of the present invention, a material capable of attenuating light energy has a property of reducing the energy of light to 1/500 or less when the light travels in the material by a distance equal to that between neighboring absorptive regions.

In a further preferred aspect of the present invention, a material capable of attenuating light energy has a property of reducing the energy of light to 1/1000 or less when the light travels in the material by a distance equal to that between neighboring absorptive regions.

In a preferred aspect of the present invention, the surface of the absorptive substrate is covered with metal, thereby forming a covering.

In a further preferred aspect of the present invention, the surface of the absorptive substrate is covered with metal or alloy selected from a group consisting of gold, silver, copper, zinc, aluminum, titanium, tantalum, chromium, iron, nickel, cobalt, lead and tin and alloys thereof, thereby forming a covering.

In the present invention, the method for covering the absorptive substrate of the biochemical analysis unit with a material capable of attenuating radiation energy and/or light energy, a material capable of attenuating radiation energy or a material capable of attenuating light energy is not particularly limited but evaporation, sputtering, chemical vapor deposition or the like is preferably employed for covering the absorptive substrate of the biochemical analysis unit with a material capable of attenuating radiation energy and/or light energy, a material capable of attenuating radiation energy or a material capable of attenuating light energy.

In a preferred aspect of the present invention, the covering of the material capable of attenuating radiation energy and/or light energy has a thickness of 0.5 to 100 times of the maximum breadth of the individual absorptive regions.

In a further preferred aspect of the present invention, the covering of the material capable of attenuating radiation energy and/or light energy has a thickness of 1 to 10 times the maximum breadth of the individual absorptive regions.

In a preferred aspect of the present invention, the plurality of absorptive regions are regularly formed in the biochemical analysis unit.

In a preferred aspect of the present invention, a plurality of absorptive regions having a substantially circular shape are formed in the biochemical analysis unit.

In another preferred aspect of the present invention, a plurality of absorptive regions having a substantially rectangular shape are formed in the biochemical analysis unit.

In a preferred aspect of the present invention, the biochemical analysis unit is formed with 10 or more absorptive regions.

In a further preferred aspect of the present invention, the biochemical analysis unit is formed with 50 or more absorptive regions.

In a further preferred aspect of the present invention, the biochemical analysis unit is formed with 100 or more absorptive regions.

In a further preferred aspect of the present invention, the biochemical analysis unit is formed with 500 or more absorptive regions.

In a further preferred aspect of the present invention, the biochemical analysis unit is formed with 1,000 or more absorptive regions.

In a further preferred aspect of the present invention, the biochemical analysis unit is formed with 5,000 or more absorptive regions.

In a further preferred aspect of the present invention, the biochemical analysis unit is formed with 10,000 or more absorptive regions.

In a further preferred aspect of the present invention, the biochemical analysis unit is formed with 50,000 or more absorptive regions.

In a further preferred aspect of the present invention, the substrate of the biochemical analysis unit is formed with 100,000 or more absorptive regions.

In a preferred aspect of the present invention, each of the plurality of absorptive regions formed in the biochemical analysis unit has a size of less than 5 mm².

In a further preferred aspect of the present invention, each of the plurality of absorptive regions formed in the biochemical analysis unit has a size of less than 1 mm².

In a further preferred aspect of the present invention, each of the plurality of absorptive regions formed in the biochemical analysis unit has a size of less than 0.5 mm².

In a further preferred aspect of the present invention, each of the plurality of absorptive regions formed in the biochemical analysis unit has a size of less than 0.1 mm².

In a further preferred aspect of the present invention, each of the plurality of absorptive regions formed in the biochemical analysis unit has a size of less than 0.05 mm².

In a further preferred aspect of the present invention, each of the plurality of absorptive regions formed in the biochemical analysis unit has a size of less than 0.01 mm².

In the present invention, the density of the absorptive regions formed in the biochemical analysis unit is determined depending upon the material covering the absorptive substrate, the thickness of the covering, the kind of electron beam released from a radioactive substance, the wavelength of fluorescence emission released from a fluorescent substance or the like.

In a preferred aspect of the present invention, the plurality of absorptive regions are formed in the biochemical analysis unit at a density of 10 or more per cm².

In a further preferred aspect of the present invention, the plurality of absorptive regions are formed in the biochemical analysis unit at a density of 50 or more per cm².

In a further preferred aspect of the present invention, the plurality of absorptive regions are formed in the biochemical analysis unit at a density of 100 or more per cm².

In a further preferred aspect of the present invention, the plurality of absorptive regions are formed in the biochemical analysis unit at a density of 500 or more per cm².

In a further preferred aspect of the present invention, the plurality of absorptive regions are formed in the biochemical analysis unit at a density of 1,000 or more per cm².

In a further preferred aspect of the present invention, the plurality of absorptive regions are formed in the biochemical analysis unit at a density of 5,000 or more per cm².

In a further preferred aspect of the present invention, the plurality of absorptive regions are formed in the biochemical analysis unit at a density of 10,000 or more per cm².

In the present invention, a porous material or a fiber material may be preferably used as the absorptive material for forming the absorptive substrate. The absorptive substrate may be formed by combining a porous material and a fiber material.

In the present invention, a porous material for forming the absorptive substrate may be any type of an organic material or an inorganic material and may be an organic/inorganic composite material.

In the present invention, an organic porous material used for forming the absorptive substrate is not particularly limited but a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof.

In the present invention, an inorganic porous material used for forming the absorptive substrate is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof.

In the present invention, a fiber material used for forming the absorptive substrate is not particularly limited. Illustrative examples of fiber materials preferably usable in the present invention include nylons such as nylon-6, nylon-6,6, nylon-4, 10; and cellulose derivatives such as nitrocellulose, acetyl cellulose, butyric-acetyl cellulose.

In a preferred aspect of the present invention, the plurality of stimulable phosphor regions of the stimulable phosphor sheet are formed by charging stimulable phosphor into a plurality of through-holes formed in the support.

In a further preferred aspect of the present invention, the plurality of stimulable phosphor regions of the stimulable phosphor sheet are formed by embedding stimulable phosphor into a plurality of through-holes formed in the support.

In a further preferred aspect of the present invention, the plurality of stimulable phosphor regions of the stimulable phosphor sheet are formed by pressing a stimulable phosphor membrane into a plurality of through-holes formed in the support.

In another preferred aspect of the present invention, the plurality of stimulable phosphor regions of the stimulable phosphor sheet are formed by charging stimulable phosphor into a plurality of recesses formed in the support.

In a further preferred aspect of the present invention, the plurality of stimulable phosphor regions of the stimulable phosphor sheet are formed by embedding stimulable phosphor into a plurality of recesses formed in the support.

In the present invention, in the case where a plurality of stimulable phosphor layer regions are formed in the stimulable phosphor sheet, the material for forming the support of the stimulable phosphor sheet may be of any type insofar as it can attenuate radiation energy. The material usable for forming the support of the stimulable phosphor sheet is not particularly limited but may be of any type of inorganic compound material or organic compound material insofar as it can attenuate radiation energy. It is preferably formed of metal material, ceramic material or plastic material.

In the present invention, illustrative examples of inorganic compound materials capable of attenuating radiation energy and preferably usable for forming the support of the stimulable phosphor sheet in the present invention include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. These may have either a monocrystal structure or a polycrystal sintered structure such as amorphous, ceramic or the like.

In the present invention, a high molecular compound is preferably used as an organic compound material preferably capable of attenuating radiation energy. Illustrative examples of high molecular compounds that are preferably usable for forming a support of the stimulable phosphor sheet in the present invention include polyolefins such as polyethylene, polypropylene and the like; is acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadiene-25 styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the capability of attenuating radiation energy generally increases as specific gravity increases, the support of the stimulable phosphor sheet is preferably formed of a compound material or a composite material having specific gravity of 1.0 g/cm³ or more and more preferably formed of a compound material or a composite material having specific gravity of 1.5 g/cm³ to 23 g/cm³.

In a preferred aspect of the present invention, a material capable of attenuating radiation energy has a property of reducing the energy of radiation to ⅕ or less when the radiation travels in the material by the distance between neighboring stimulable phosphor layer regions.

In a further preferred aspect of the present invention, the support of the stimulable phosphor sheet is made of a material capable of reducing the energy of radiation to 1/10 or less when the radiation travels in the material by the distance between neighboring stimulable phosphor layer regions.

In a further preferred aspect of the present invention, the support of the stimulable phosphor sheet is made of a material capable of reducing the energy of radiation to 1/50 or less when the radiation travels in the material by the distance between neighboring stimulable phosphor layer regions.

In a further preferred aspect of the present invention, the support of the stimulable phosphor sheet is made of a material capable of reducing the energy of radiation to 1/100 or less when the radiation travels in the material by the distance between neighboring stimulable phosphor layer regions.

In a further preferred aspect of the present invention, the support of the stimulable phosphor sheet is made of a material capable of reducing the energy of radiation to 1/500 or less when the radiation travels in the material by the distance between neighboring stimulable phosphor layer regions.

In a further preferred aspect of the present invention, the support of the stimulable phosphor sheet is made of a material capable of reducing the energy of radiation to 1/1000 or less when the radiation travels in the material by the distance between neighboring stimulable phosphor layer regions.

In the present invention, the stimulable phosphor usable for storing radiation energy may be of any type insofar as it can store radiation energy or electron beam energy and can be stimulated by an electromagnetic wave to release the radiation energy or the electron beam energy stored therein in the form of light. More specifically, preferably employed stimulable phosphors include alkaline earth metal fluorohalide phosphors (Ba₁₋ₓ, M²⁺ₓ)FX:yA (where M²⁺ is at least one alkaline earth metal selected from the group consisting of Mg, Ca, Sr, Zn and Cd; X is at least one element selected from the group consisting of Cl, Br and I, A is at least one element selected from the group consisting of Eu, Tb, Ce, Tm, Dy, Pr, Ho, Nd, Yb and Er; x is equal to or greater than 0 and equal to or less than 0.6 and y is equal to or greater than 0 and equal to or less than 0.2) disclosed in U.S. Pat. No. 4,239,968, alkaline earth metal fluorohalide phosphors SrFX:Z (where X is at least one halogen selected from the group consisting of Cl, Br and I; Z is at least one of Eu and Ce) disclosed in Japanese Patent Application Laid Open No. 2-276997, europium activated complex halide phosphors BaFXxNaX′:aEu²⁺ (where each of X or X′ is at least one halogen selected from the group consisting of Cl, Br and I; x is greater than 0 and equal to or less than 2; and y is greater than 0 and equal to or less than 0.2) disclosed in Japanese Patent Application Laid Open No. 59-56479, cerium activated trivalent metal oxyhalide phosphors MOX:xCe (where M is at least one trivalent metal selected from the group consisting of Pr, Nd, Pm, Sm, Eu, Tb, Dy, Ho, Er, Tm, Yb and Bi; X is at least one halogen selected from the group consisting of Br and I; and x is greater than 0 and less than 0.1) disclosed in Japanese Patent Application laid Open No. 58-69281, cerium activated rare earth oxyhalide phosphors LnOX:xCe (where Ln is at least one rare earth element selected from the group consisting of Y, La, Gd and Lu; X is at least one halogen selected from the group consisting of Cl, Br and I; and x is greater than 0 and equal to or less than 0.1) disclosed in U.S. Pat. No. 4,539,137, and europium activated complex halide phosphors M^{II}FXaM^{I}X′bM′^{II}X″₂cM^{III}X″₃xA:yEu²⁺ (where M^{II} is at least one alkaline earth metal selected from the group consisting of Ba, Sr and Ca; M^{I} is at least one alkaline metal selected from the group consisting of Li, Na, K, Rb and Cs; M′^{II} is at least one divalent metal selected from the group consisting of Be and Mg; M^{III} is at least one trivalent metal selected from the group consisting of Al, Ga, In and Ti; A is at least one metal oxide; X is at least one halogen selected from the group consisting of Cl, Br and I; each of X′, X″ and X′″ is at least one halogen selected from the group consisting of F, Cl, Br and I; a is equal to or greater than 0 and equal to or less than 2; b is equal to or greater than 0 and equal to or less than 10-2; c is equal to or greater than 0 and equal to or less than 10-2; a+b+c is equal to or greater than 10-2; x is greater than 0 and equal to or less than 0.5; and y is greater than 0 and equal to or less than 0.2) disclosed in U.S. Pat. No. 4,962,047.

In the present invention, the stimulable phosphor usable for storing the energy of chemiluminescence emission may be of any type insofar as it can store the energy of light in the wavelength band of visible light and can be stimulated by an electromagnetic wave to release in the form of light the energy of light in the wavelength band of visible light stored therein. More specifically, preferably employed stimulable phosphors include at least one selected from the group consisting of metal halophosphates, rare-earth-activated sulfide-host phosphors, aluminate-host phosphors, silicate-host phosphors, fluoride-host phosphors and mixtures of two, three or more of these phosphors. Among them, rare-earth-activated sulfide-host phosphors are more preferable and, particularly, rare-earthactivated alkaline earth metal sulfide-host phosphors disclosed in U.S. Pat. Nos. 5,029,253 and 4,983,834, zinc germanate such as Zn₂GeO₄:Mn, V; Zn₂GeO₄:Mn disclosed in Japanese Patent Application Laid Open No. 2001-131545, alkaline-earth aluminate such as Sr₄Al₁₄O₂₅:Ln (wherein Ln is a rare-earth element) disclosed in Japanese Patent Application Laid Open No. 2001-123162, zinc silicate such as Y_{0.8}Lu_{1.2}SiO₅:Ce, Zr; GdOCl:Ce disclosed in Japanese Patent Publication No. 6-31904 and the like are most preferable.

The above and other objects and features of the present invention will become apparent from the following description made with reference to the accompanying drawings.

### BRIEF DESCRIPTION OF THE DRAWINGS

FIG. 1 is a schematic perspective view showing a biochemical analysis unit which is a preferred embodiment of the present invention.

FIG. 2 is a schematic partial cross-sectional view of a biochemical analysis unit.

FIG. 3 is a schematic front view showing a spotting device.

FIG. 4 is a schematic front view showing a hybridization reaction vessel.

FIG. 5 is a schematic cross-sectional view showing a method for exposing a stimulable phosphor layer formed on a stimulable phosphor sheet by a radioactive labeling substance contained in absorptive regions.

FIG. 6 is a schematic view showing one example of a scanner.

FIG. 7 is a schematic perspective view showing details in the vicinity of a photomultiplier.

FIG. 8 is a schematic cross-sectional view taken along a line A-A in FIG. 7.

FIG. 9 is a schematic cross-sectional view taken along a line B-B in FIG. 7.

FIG. 10 is a schematic cross-sectional view taken along a line C-C in FIG. 7.

FIG. 11 is a schematic cross-sectional view taken along a line D-D in FIG. 7.

FIG. 12 is a schematic plan view of a scanning mechanism of an optical head.

FIG. 13 is a block diagram of a control system, an input system and a drive system of a scanner shown in FIG. 6.

FIG. 14 is a schematic front view showing a data producing system for reading chemiluminescent data of a labeling substance which generates chemiluminescent emission when it contacts a chemiluminescent substrate and producing biochemical analysis data.

FIG. 15 is a schematic longitudinal cross sectional view showing a cooled CCD camera.

FIG. 16 is a schematic vertical cross sectional view showing a dark box.

FIG. 17 is a block diagram of a personal computer and peripheral devices thereof.

FIG. 18 is a schematic perspective view showing a stimulable phosphor sheet used in a biochemical analyzing method according to another preferred embodiment of the present invention.

FIG. 19 is a schematic cross-sectional view showing a method for exposing a number of stimulable phosphor layer regions formed on a stimulable phosphor sheet shown in FIG. 18 by a radioactive labeling substance contained in a number of absorptive regions.

FIG. 20 is a schematic perspective view showing a stimulable phosphor sheet used in a biochemical analyzing method according to a further preferred embodiment of the present invention.

FIG. 21 is a schematic cross-sectional view showing a method for exposing a number of stimulable phosphor layer regions formed on a stimulable phosphor sheet shown in FIG. 20 by a radioactive labeling substance contained in a number of absorptive regions.

### DESCRIPTION OF THE PREFERRED EMBODIMENTS

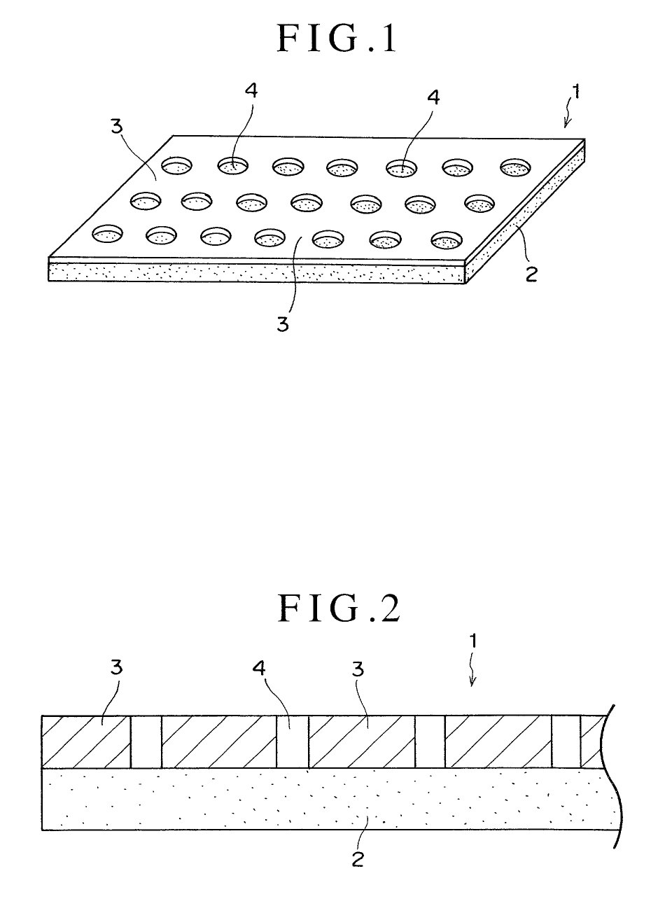

FIG. 1 is a schematic perspective view showing a biochemical analysis unit which is a preferred embodiment of the present invention and FIG. 2 is a schematic partial cross-sectional view thereof.

As shown in FIGS. 1 and 2, a biochemical analysis unit 1 includes an absorptive substrate 2 formed of nylon 6 and gold is vapor deposited onto the surface of the absorptive substrate 2 via a mask, to form vapor-deposited gold regions. The absorptive substrate is exposed to the outside at regions where no vapor-deposited gold region is formed, whereby a number of absorptive regions 4 are dot-like formed.

Although not accurately shown in FIGS. 1 and 2, in this embodiment, the covering region 3 of gold is formed on the surface of the absorptive substrate 2 so that absorptive regions having a size of about 0.07 cm² are regularly formed in a matrix manner of 120 columns×160 lines and, therefore, 19,200 absorptive regions 4 are formed.

In this embodiment, gold is vapor deposited so that the thickness of the covering region 3 of gold is about double the diameter of the absorptive region 4.

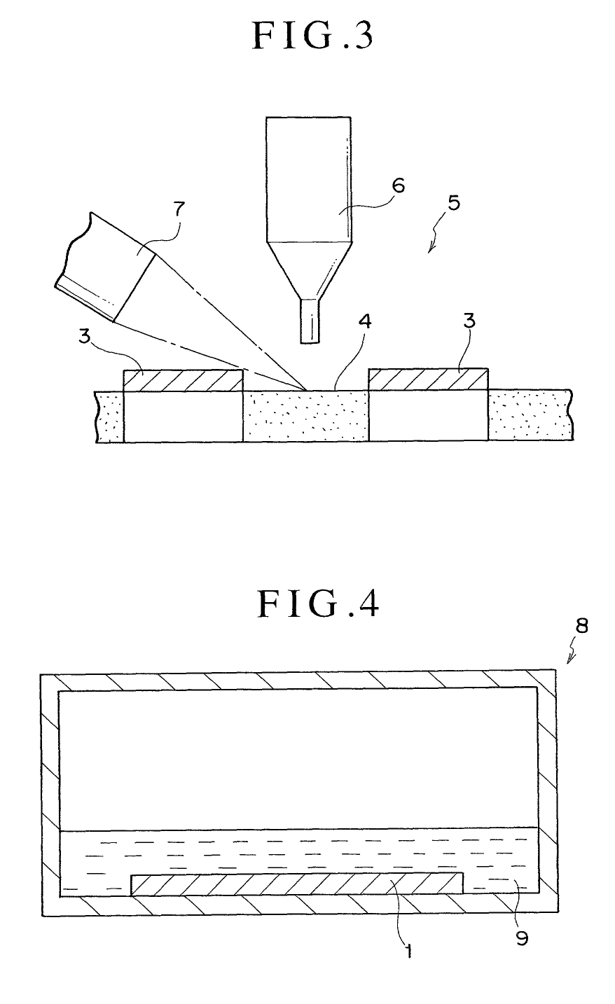

FIG. 3 is a schematic front view showing a spotting device.

As shown in FIG. 3, when biochemical analysis is performed, a solution containing specific binding substances such as a plurality of cDNAs whose sequences are known but are different from each other are spotted using a spotting device 5 onto a number of the absorptive regions 4 of the biochemical analysis unit 1 and fixed therein.

As shown in FIG. 3, the spotting device 5 includes an injector 6 for ejecting a solution of specific binding substances toward the biochemical analysis unit 1 and a CCD camera 7 and is constituted so that the solution of specific binding substances such as cDNAs are spotted from the injector 6 when the tip end portion of the injector 6 and the center of the absorptive region 4 into which the solution of a specific binding substance is to be spotted are determined to coincide with each other as a result of viewing them using the CCD camera, thereby ensuring that the solution of specific binding substances can be accurately spotted into a number of the absorptive regions 4 of the biochemical analysis unit 1.

FIG. 4 is a schematic front view showing a hybridization reaction vessel.

As shown in FIG. 4, a hybridization reaction vessel 8 is formed to have a substantially rectangular cross section and accommodates a hybridization solution 9 containing a substance derived from a living organism labeled with a labeling substance therein.

In the case where a specific binding substance such as cDNA is to be labeled with a radioactive labeling substance, a hybridization solution 9 containing a substance derived from a living organism labeled with a radioactive labeling substance is prepared and is accommodated in the hybridization reaction vessel 8.

On the other hand, in the case where a specific binding substance such as cDNA is to be labeled with a labeling substance which generates chemiluminescent emission when it contacts a chemiluminescent substrate, a hybridization solution 9 containing a substance derived from a living organism labeled with a labeling substance which generates chemiluminescent emission when it contacts a chemiluminescent substrate is prepared and is accommodated in the hybridization reaction vessel 8.

Further, in the case where a specific binding substance such as cDNA is to be labeled with a fluorescent substance such as a fluorescent dye, a hybridization solution 9 containing a substance derived from a living organism labeled with a fluorescent substance such as a fluorescent dye is prepared and is accommodated in the hybridization reaction vessel 8.

It is possible to prepare a hybridization solution 9 containing two or more substances derived from a living organism among a substance derived from a living organism labeled with a radioactive labeling substance, a substance derived from a living organism labeled with a labeling substance which generates chemiluminescent emission when it contacts a chemiluminescent substrate and a substance derived from a living organism labeled with a fluorescent substance such as a fluorescent dye and accommodate it in the hybridization vessel 8. In this embodiment, a hybridization solution 9 containing a substance derived from a living organism labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate is prepared and accommodated in the hybridization reaction vessel 8.

When hybridization is to be performed, the biochemical analysis unit 1 containing specific binding substances such as a plurality of cDNAs spotted into a number of absorptive regions 4 is accommodated in the hybridization reaction vessel 8.

As a result, specific binding substances spotted in a number of the absorptive regions 4 of the biochemical analysis unit 1 can be selectively hybridized with a substance derived from a living organism labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate.

In this manner, fluorescence data of a fluorescent substance such as a fluorescent dye and chemiluminescence data of a substance derived from a living organism labeled with a labeling substance which generates chemiluminescent emission when it contacts a chemiluminescent substrate are recorded in a number of absorptive regions 4 formed in the biochemical analysis unit 1. Fluorescence data recorded in the biochemical analysis unit 1 are read by a scanner described later, thereby producing biochemical analysis data and chemiluminescence data recorded in the biochemical analysis unit 1 are read by a data producing system described later, thereby producing biochemical analysis data.

On the other hand, radiation data of the radioactive labeling substance recorded in a number of absorptive regions 4 formed in the biochemical analysis unit 1 are transferred onto a stimulable phosphor layer of a stimulable phosphor sheet and read by the scanner described later, thereby producing biochemical analysis data.

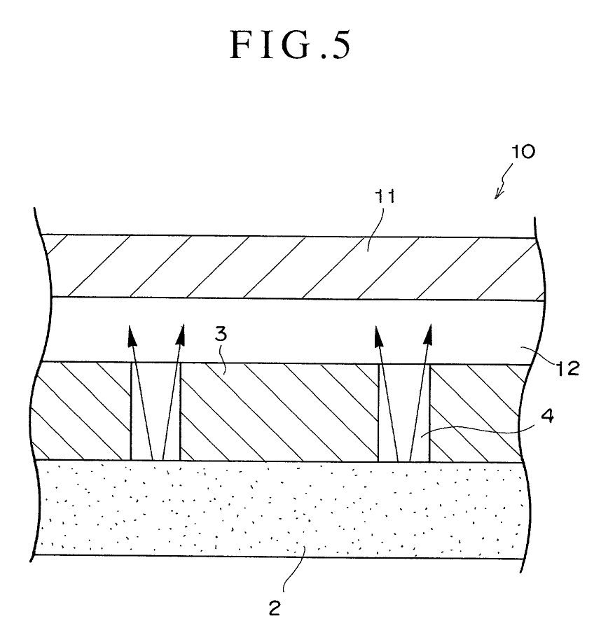

FIG. 5 is a schematic cross-sectional view showing a method for exposing a stimulable phosphor layer formed on a stimulable phosphor sheet to a radioactive labeling substance contained in a number of absorptive regions 4 of the biochemical analysis unit 1.

As shown in FIG. 5, when a stimulable phosphor layer 12 of a stimulable phosphor sheet 10 is to be exposed, the stimulable phosphor sheet 10 is superposed on the biochemical analysis unit 1 in such a manner that the stimulable phosphor layer 12 uniformly formed on one surface of a support 11 of the stimulable phosphor sheet 10 abuts against the covering regions 3 of gold of the biochemical analysis unit 1.

During the exposure operation, electron beams (P rays) are released from the radioactive labeling substance contained in the absorptive regions 4 of the biochemical analysis unit 1. However, since the vapor-deposited gold regions 3 are formed on the surface of the absorptive substrate 2 and neighboring absorptive regions 4 are isolated by the covering regions 3 of gold, electron beams (P rays) released from the radioactive labeling substance contained in each of the absorptive regions 4 can be effectively prevented from entering regions of the stimulable phosphor layer 12 other than a region each of the absorptive regions 4 faces and, therefore, it is possible to selectively expose only the region of the stimulable phosphor layer 12 each of the absorptive regions 4 faces to the electron beams (β rays) released from the radioactive labeling substance contained in each of the absorptive regions 4.

Further, in this embodiment, since gold is vapor deposited onto the surface of the absorptive substrate 2 so that the thickness of the covering region 3 of gold is about double the diameter of the individual absorptive regions 4, the electron beams (β rays) released from the radioactive labeling substance contained in each of the absorptive regions 4 are prevented by the collimation effect from broadening and, therefore, it is possible to selectively expose only the region of the stimulable phosphor layer 12 each of the absorptive regions 4 faces to the electron beams (β rays) released from the radioactive labeling substance contained in each of the absorptive regions 4.

In this manner, radiation data of a radioactive labeling substance are recorded in the stimulable phosphor layer 12 formed on the support 11 of the stimulable phosphor sheet 10.

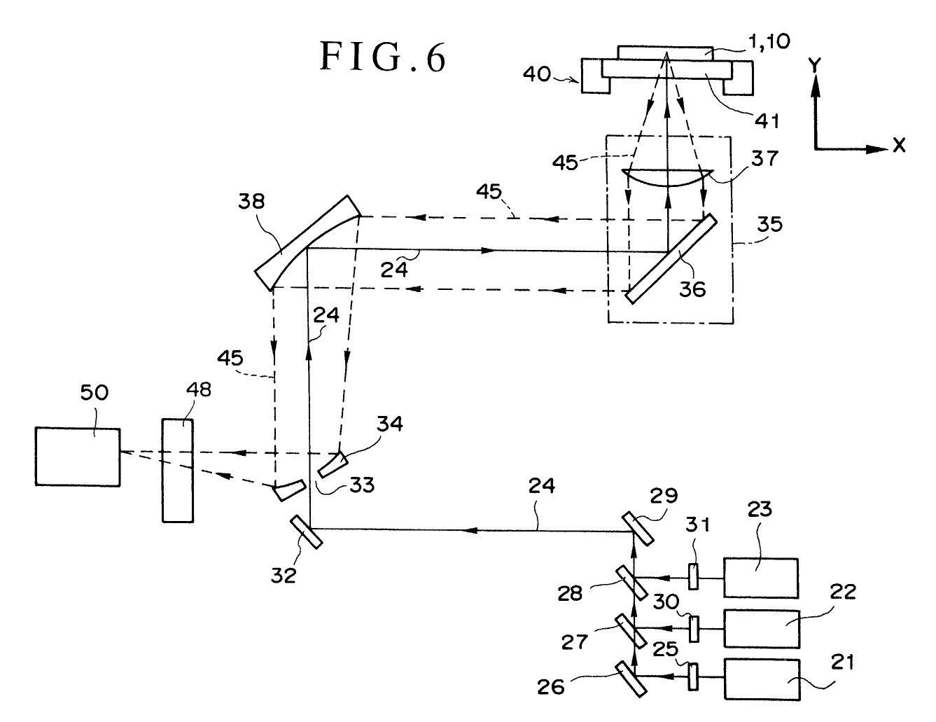

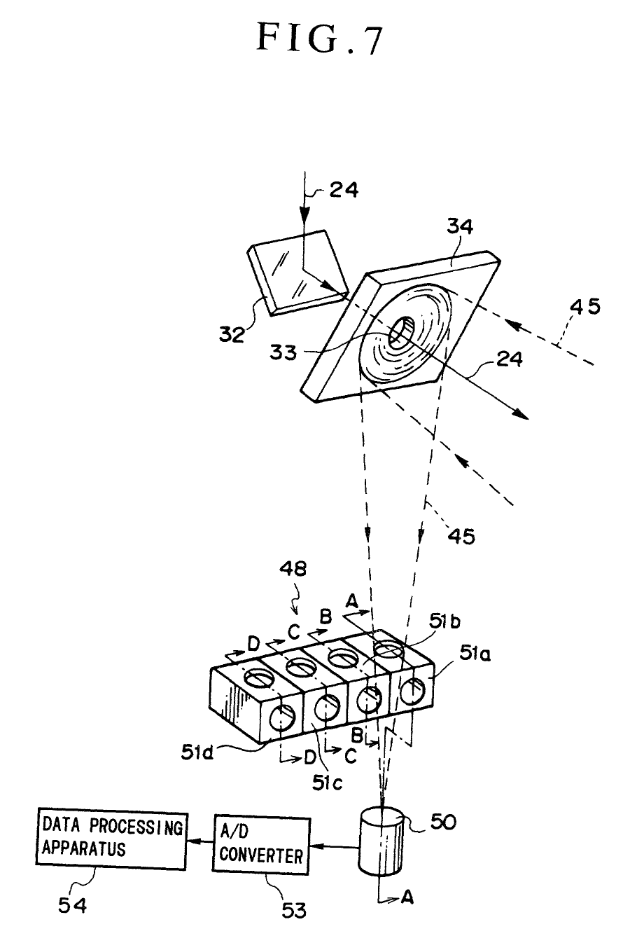

FIG. 6 is a schematic view showing one example of a scanner for reading radiation data of a radioactive labeling substance recorded in the stimulable phosphor layer 12 formed on the support of the stimulable phosphor sheet 10 and fluorescence data recorded in a number of the absorptive regions 4 formed in the biochemical analysis unit 1 and producing biochemical analysis data, and FIG. 7 is a schematic perspective view showing details in the vicinity of a photomultiplier.

The scanner shown in FIG. 6 is constituted so as to read radiation data of a radioactive labeling substance recorded in the stimulable phosphor layer 12 formed on the support 11 of the stimulable phosphor sheet 10 and fluorescence data recorded in a number of the absorptive regions 4 of the biochemical analysis unit 1 and includes a first laser stimulating ray source 21 for emitting a laser beam having a wavelength of 640 nm, a second laser stimulating ray source 22 for emitting a laser beam having a wavelength of 532 nm and a third laser stimulating ray source 23 for emitting a laser beam having a wavelength of 473 nm. In this embodiment, the first laser stimulating ray source 21 is constituted by a semiconductor laser beam source and the second laser stimulating ray source 22 and the third laser stimulating ray source are constituted by a second harmonic generation element.

A laser beam 24 emitted from the first laser stimulating source 21 passes through a collimator lens 25, thereby being made a parallel beam, and is reflected by a mirror 26. A first dichroic mirror for transmitting light having a wavelength of 640 nm but reflecting light having a wavelength of 532 nm and a second dichroic mirror 28 for transmitting light having a wavelength equal to and longer than 532 nm but reflecting light having a wavelength of 473 nm are provided in the optical path of the laser beam 24 emitted from the first laser stimulating ray source 21. The laser beam 24 emitted from the first laser stimulating ray source 21 and reflected by the mirror 26 passes through the first dichroic mirror 27 and the second dichroic mirror 28 and advances to a mirror 29.

On the other hand, the laser beam 24 emitted from the second laser stimulating ray source 22 passes through a collimator lens 30, thereby being made a parallel beam, and is reflected by the first dichroic mirror 27, thereby changing its direction by 90 degrees. The laser beam 24 then passes through the second dichroic mirror 28 and advances to the mirror 29.

Further, the laser beam 24 emitted from the third laser stimulating ray source 23 passes through a collimator lens 31, thereby being made a parallel beam, and is reflected by the second dichroic mirror 28, thereby changing its direction by 90 degrees. The laser beam 24 then advances to the mirror 29.

The laser beam 24 advancing to the mirror 29 is reflected by the mirror 29 and advances to a mirror 32 to be reflected thereby.

A perforated mirror 34 formed with a hole 33 at the center portion thereof is provided in the optical path of the laser beam 24 reflected by the mirror 32. The laser beam 24 reflected by the mirror passes through the hole 33 of the perforated mirror 34 and advances to a concave mirror 38.

The laser beam 24 advancing to the concave mirror 38 is reflected by the concave mirror 38 and enters an optical head 35.

The optical head 35 includes a mirror 36 and an aspherical lens 37. The laser beam 24 entering the optical head 35 is reflected by the mirror 36 and condensed by the aspherical lens 37 onto the stimulable phosphor sheet 10 or the biochemical analysis unit 1 placed on the glass plate 41 of a stage 40.

When the laser beam 24 impinges on the stimulable phosphor layer 12 of the stimulable phosphor 10, stimulable phosphor contained in the stimulable phosphor layer 12 formed on the support of the stimulable phosphor 10 is excited, thereby releasing stimulated emission 45. On the other hand, when the laser beam 24 impinges on the biochemical analysis unit 1, a fluorescent dye or the like contained in the absorptive region 4 of the biochemical analysis unit 1 is excited, thereby releasing fluorescence emission 45.

The stimulated emission 45 released from the stimulable phosphor layer 12 of the stimulable phosphor 10 or the fluorescence emission 45 released from the absorptive region 4 of the biochemical analysis unit 1 is condensed onto the mirror 36 by the aspherical lens 37 provided in the optical head 35 and reflected by the mirror on the side of the optical path of the laser beam 24, thereby being made a parallel beam to advance to the concave mirror 38.

The stimulated emission 45 or the fluorescence emission 45 advancing to the concave mirror 38 is reflected by the concave mirror and advances to the perforated mirror 34.

As shown in FIG. 7, the stimulated emission 45 or the fluorescence emission 45 advancing to the perforated mirror 34 is reflected downward by the perforated mirror 34 formed as a concave mirror and advances to a filter unit 48, whereby light having a predetermined wavelength is cut. The stimulated emission 45 or the fluorescence emission 45 then impinges on a photomultiplier 50, thereby being photoelectrically detected.

As shown in FIG. 7, the filter unit 48 is provided with four filter members 51a, 51b, 51c and 51d and is constituted to be laterally movable in FIG. 7 by a motor (not shown).

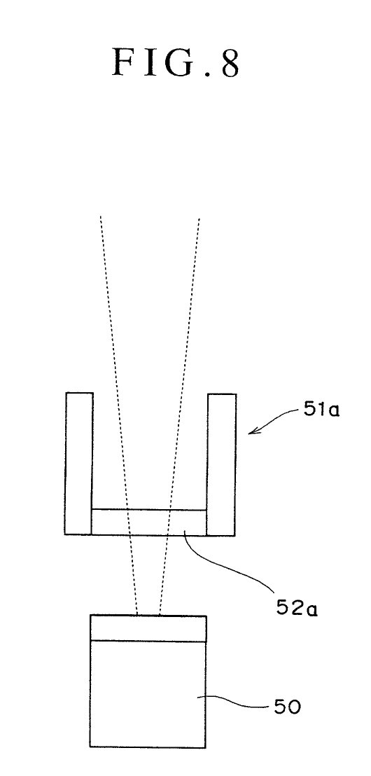

FIG. 8 is a schematic cross-sectional view taken along a line A-A in FIG. 7.

As shown in FIG. 8, the filter member 51 a includes a filter 52a and the filter 52a is used for reading fluorescence emission 45 by stimulating a fluorescent substance such as a fluorescent dye contained in a number of the absorptive regions 4 of the biochemical analysis unit 1 using the first laser stimulating ray source 21 and has a property of cutting off light having a wavelength of 640 nm but transmitting light having a wavelength longer than 640 nm.

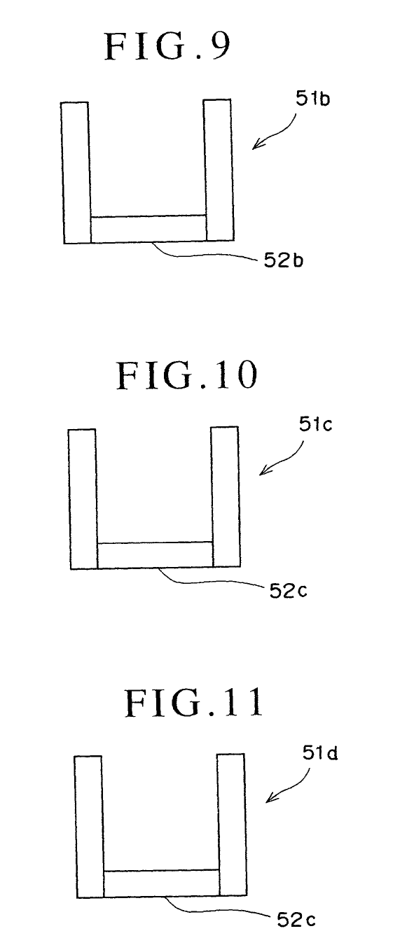

FIG. 9 is a schematic cross-sectional view taken along a line B-B in FIG. 7.

As shown in FIG. 9, the filter member 51b includes a filter 52b and the filter 52b is used for reading fluorescence emission 45 by stimulating a fluorescent substance such as a fluorescent dye contained in number of the absorptive regions 4 of the biochemical analysis unit 1 using the second laser stimulating ray source 22 and has a property of cutting off light having a wavelength of 532 nm but transmitting light having a wavelength longer than 532 nm.

FIG. 10 is a schematic cross-sectional view taken along a line C-C in FIG. 7.

As shown in FIG. 10, the filter member 51c includes a filter 52c and the filter 52c is used for reading fluorescence emission 45 by stimulating a fluorescent substance such as a fluorescent dye contained in in a number of the absorptive regions 4 of the biochemical analysis unit 1 using the third laser stimulating ray source 23 and has a property of cutting off light having a wavelength of 473 nm but transmitting light having a wavelength longer than 473 nm.

FIG. 11 is a schematic cross-sectional view taken along a line D-D in FIG. 7.

As shown in FIG. 11, the filter member 51 d includes a filter 52d and the filter 52d is used for reading stimulated emission released from stimulable phosphor contained in the stimulable phosphor layer 12 formed on the support 11 of the stimulable phosphor sheet 10 upon being stimulated using the first laser stimulating ray source 1 and has a property of transmitting only light having a wavelength corresponding to that of stimulated emission emitted from stimulable phosphor but cutting off light having a wavelength of 640 nm.

Therefore, in accordance with the kind of a stimulating ray source to be used, one of these filter members 51a, 51b, 51c, 51d is selectively positioned in front of the photomultiplier 50, thereby enabling the photomultiplier 50 to photoelectrically detect only light to be detected.

The analog data produced by photoelectrically detecting light with the photomultiplier 50 are converted by an A/D converter 53 into digital data and the digital data are fed to a data processing apparatus 54.

Although not shown in FIG. 6, the optical head 35 is constituted to be movable by a scanning mechanism in the X direction and the Y direction in FIG. 6 so that the whole surface of the stimulable phosphor layer 12 formed on the support 11 of the stimulable phosphor sheet 10 or all of the absorptive regions 4 formed in the biochemical analysis unit 1 can be scanned by the laser beam 24.

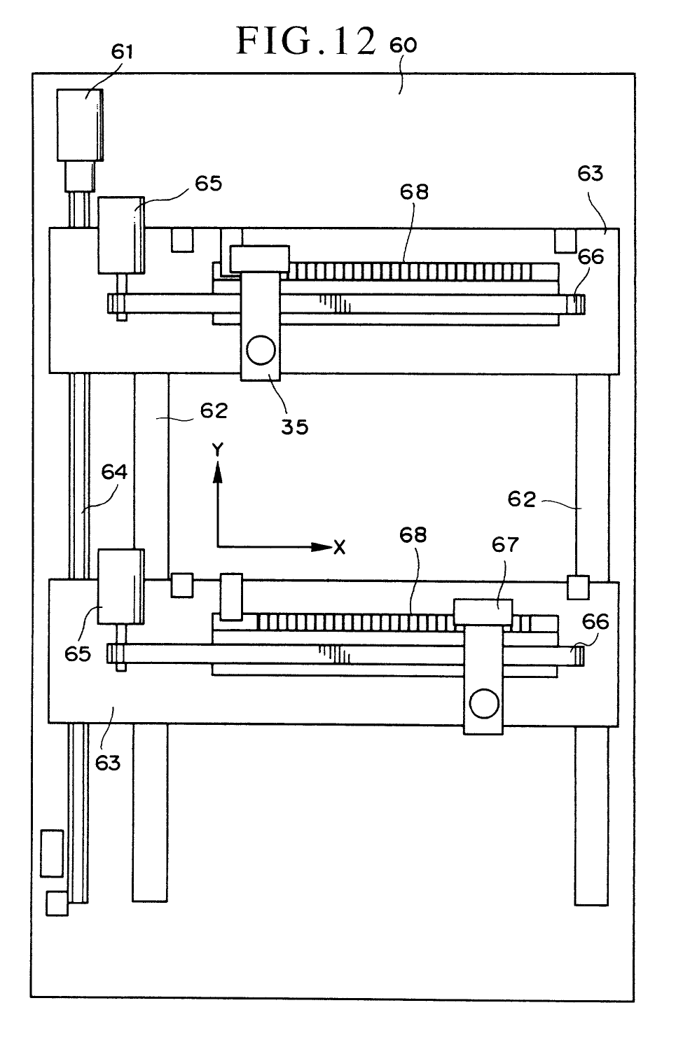

FIG. 12 is a schematic plan view showing the scanning mechanism of the optical head 35. In FIG. 12, optical systems other than the optical head 35 and the paths of the laser beam 24 and stimulated emission 45 or fluorescence emission 45 are omitted for simplification.

As shown in FIG. 12, the scanning mechanism of the optical head 35 includes a base plate 60, and a sub-scanning pulse motor and a pair of rails 62, 62 are fixed on the base plate 60. A movable base plate 63 is further provided so as to be movable in the sub-scanning direction indicated by an arrow Y in FIG. 12.

The movable base plate 63 is formed with a threaded hole (not shown) and a threaded rod 64 rotated by the sub-scanning pulse motor 61 is engaged with the inside of the hole.

A main scanning pulse motor 65 is provided on the movable base plate 63. The main scanning pulse motor 65 is adapted for intermittently driving an endless belt 66 by the pitch equal to the distance between the neighboring absorptive regions 4 formed in the biochemical analysis unit 1. The optical head 35 is fixed to the endless belt 66 and when the endless belt 66 is driven by the main scanning pulse motor 65, the optical head 35 is moved in the main scanning direction indicated by an arrow X in FIG. 12. In FIG. 12, the reference numeral 67 designates a linear encoder for detecting the position of the optical head 35 in the main scanning direction and the reference numeral 68 designates slits of the linear encoder 67.

Therefore, the optical head 35 is moved in the X direction and the Y direction in FIG. 12 by driving the endless belt 66 in the main scanning direction by the main scanning pulse motor 65 and intermittently moving the movable base plate 63 in the sub-scanning direction by the sub-scanning pulse motor 61, thereby scanning the whole surface of the stimulable phosphor layer 12 formed on the support 11 of the stimulable phosphor sheet 10 or all of the absorptive regions 4 formed in the biochemical analysis unit 1 with the laser beam 24.

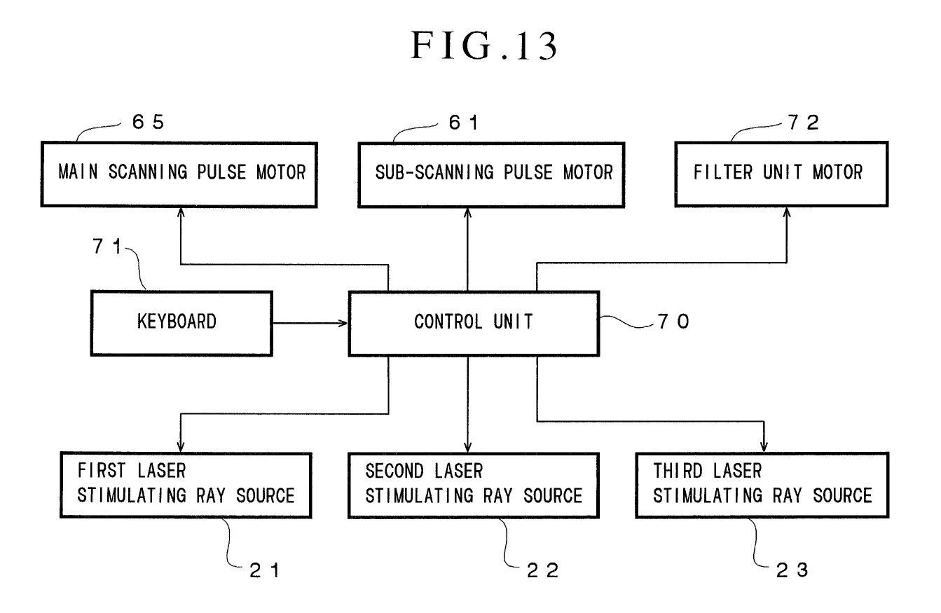

FIG. 13 is a block diagram of a control system, an input system and a drive system of the scanner shown in FIG. 6.

As shown in FIG. 13, the control system of the scanner includes a control unit 70 for controlling the whole operation of the scanner and the input system of the scanner includes a keyboard 71 which can be operated by a user and through which various instruction signals can be input.

As shown in FIG. 13, the drive system of the scanner includes the main scanning pulse motor 65 for moving the optical head 35 in the main scanning direction, the sub-scanning pulse motor 61 for moving the optical head 35 in the sub-scanning direction and a filter unit motor 72 for moving the filter unit 48 provided with the four filter members 51a, 51b, 51c and 51d.

The control unit 70 is adapted for selectively outputting a drive signal to the first laser stimulating ray source 21, the second laser stimulating ray source 22 or the third laser stimulating ray source 23 and outputting a drive signal to the filter unit motor 72.

The thus constituted scanner reads radiation data recorded in a stimulable phosphor sheet 10 by exposing the stimulable phosphor layer 12 to a radioactive labeling substance contained in a number of the absorptive regions 4 formed in the biochemical analysis unit 1 and produces biochemical analysis data in the following manner.

A stimulable phosphor sheet 10 is first set on the glass plate of the stage 40 by a user.

An instruction signal indicating that radiation data recorded in the stimulable phosphor layer 12 formed on the support 11 of the stimulable phosphor sheet 10 are to be read is then input through the keyboard 71.

The instruction signal input through the keyboard 71 is input to the control unit 70 and the control unit 70 outputs a drive signal to the filter unit motor 72 in accordance with the instruction signal, thereby moving the filter unit 48 so as to locate the filter member 51d provided with the filter 52d having a property of transmitting only light having a wavelength corresponding to that of stimulated emission emitted from stimulable phosphor but cutting off light having a wavelength of 640 nm in the optical path of stimulated emission 45.

The control unit 70 then outputs a drive signal to the first laser stimulating ray source 21 to activate it, thereby causing it to emit a laser beam 24 having a wavelength of 640 nm.

The laser beam 24 emitted from the first laser stimulating ray source 21 is made a parallel beam by the collimator lens 25 and advances to the mirror 26 to be reflected thereby.

The laser beam 24 reflected by the mirror 26 passes through the first dichroic mirror 27 and the second dichroic mirror 28 and advances to the mirror 29.

The laser beam 24 advancing to the mirror 29 is reflected by the mirror 29 and further advances to a mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto the stimulable phosphor layer 12 of the stimulable phosphor sheet 10 placed on the glass plate 41 of the stage 40.

As a result, a stimulable phosphor contained in the stimulable phosphor layer 12 formed on the support 11 of the stimulable phosphor sheet 10 is stimulated by the laser beam 24 and stimulated emission 45 is released from the stimulable phosphor.

The stimulated emission 45 released from the stimulable phosphor contained in the stimulable phosphor layer 12 is condensed by the aspherical lens 37 provided in the optical head 35 and reflected by the mirror 36 on the side of an optical path of the laser beam 24, thereby being made a parallel beam to advance to the concave mirror 38.

The stimulated emission 45 advancing to the concave mirror is reflected by the concave mirror 38 and advances to the perforated mirror 34.

As shown in FIG. 7, the stimulated emission 45 advancing to the perforated mirror 34 is reflected downward by the perforated mirror 34 formed as a concave mirror and advances to the filter 52d of the filter unit 48.

Since the filter 52d has a property of transmitting only light having a wavelength corresponding to that of stimulated emission emitted from stimulable phosphor but cutting off light having a wavelength of 640 nm, light having a wavelength of 640 nm corresponding to that of the stimulating ray is cut off by the filter 52d and only light having a wavelength corresponding to that of stimulated emission passes through the filter 52d to be photoelectrically detected by the photomultiplier 50.

As described above, since the optical head 35 is moved on the base plate 63 in the X direction in FIG. 12 by the main scanning pulse motor 65 mounted on the base plate 63 and the base plate 63 is moved in the Y direction in FIG. 12 by the sub-scanning pulse motor 61, the whole surface of the stimulable phosphor layer 12 formed on the support 11 of the stimulable phosphor sheet 10 is scanned by the laser beam 24. Therefore, the photomultiplier 50 can read radiation data of a radioactive labeling substance recorded in the stimulable phosphor layer 12 by photoelectrically detecting the stimulated emission 45 released from stimulable phosphor contained in the stimulable phosphor layer 12 and produce analog data for biochemical analysis.

The analog data produced by photoelectrically detecting the stimulated emission 45 with the photomultiplier 50 are converted by the A/D converter 53 into digital data and the digital data are fed to the data processing apparatus 54.

On the other hand, when fluorescence data of a fluorescent substance such as a fluorescent dye carried in a number of the absorptive regions 4 formed in the biochemical analysis unit 1 are to be read to produce biochemical analysis data, a biochemical analysis unit 1 is first set on the glass plate 41 of the stage 40 by a user.

A fluorescent substance identification signal for identifying the kind of fluorescent substance as a labeling substance is then input through the keyboard 71 by the user together with an instruction signal indicating that fluorescence data are to be read.

The fluorescent substance identification signal and the instruction signal are input to the control unit 70 and when the control unit 70 receives them, it determines the laser stimulating ray source to be used in accordance with a table stored in a memory (not shown) and also determines what filter is to be positioned in the optical path of fluorescence emission 45 among the filters 52a, 52b and 52c.

For example, when Rhodamine (registered trademark), which can be most efficiently stimulated by a laser beam having a wavelength of 532 nm, is used as a fluorescent substance for labeling a substance derived from a living organism and a signal indicating such a fact is input, the control unit 70 selects the second laser stimulating ray source 22 and the filter 52b and outputs a drive signal to the filter unit motor 72, thereby moving the filter unit 48 so that the filter member 51b inserting the filter 52b having a property of cutting off light having a wavelength of 532 nm but transmitting light having a wavelength longer than 532 nm in the optical path of the fluorescence emission 45.

The control unit 70 then outputs a drive signal to the second laser stimulating ray source 22 to activate it, thereby causing it to emit a laser beam 24 having a wavelength of 532 nm.

The laser beam 24 emitted from the second laser stimulating ray source 22 is made a parallel beam by the collimator lens 30, advances to the first dichroic mirror 27 and is reflected thereby.

The laser beam 24 reflected by the first dichroic mirror 27 transmits through the second dichroic mirror 28 and advances to the mirror 29.

The laser beam 24 advancing to the mirror 29 is reflected by the mirror 29 and further advances to the mirror 32 to be reflected thereby.

The laser beam 24 reflected by the mirror 32 advances to the perforated mirror 34 and passes through the hole 33 of the perforated mirror 34. Then, the laser beam 24 advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto one of the absorptive regions 4 of the biochemical analysis unit 1 placed on the glass plate 41 of the stage 40.

In this embodiment, since a number of the absorptive regions are isolated from each other by the covering regions 3 of gold, it is possible to efficiently prevent a laser beam 24 entering the absorptive region 4 from scattering and stimulating a fluorescent substance contained in neighboring absorptive regions.

When the laser beam 24 enters an absorptive region 4 formed in the biochemical analysis unit 1, a fluorescent substance such as a fluorescent dye, for instance, Rhodamine, contained in the absorptive region 4 formed in the biochemical analysis unit 1 is stimulated by the laser beam 24 and fluorescence emission 45 is released from Rhodamine.

In the biochemical analysis unit 1 according to this embodiment, since the covering regions 3 of gold are formed on the absorptive substrate 2 and neighboring absorptive regions 4 are isolated by the covering regions 3 of gold, it is possible to reliably prevent fluorescence emission released from a fluorescent substance contained in an absorptive region 4 from being mixed with fluorescent released from a fluorescent substance contained in the neighboring absorptive regions 4.

The fluorescence emission 45 released from Rhodamine is condensed by the aspherical lens 37 provided in the optical head 35 and reflected by the mirror 36 on the side of an optical path of the laser beam 24, thereby being made a parallel beam to advance to the concave mirror 38.

The fluorescence emission 45 advancing to the concave mirror is reflected by the concave mirror 38 and advances to the perforated mirror 34.

As shown in FIG. 7, the fluorescence emission 45 advancing to the perforated mirror 34 is reflected downward by the perforated mirror 34 formed as a concave mirror and advances to the filter 52b of a filter unit 48.

Since the filter 52b has a property of cutting off light having a wavelength of 532 nm but transmitting light having a wavelength longer than 532 nm, light having the same wavelength of 532 nm as that of the stimulating ray is cut off by the filter 52b and only light in the wavelength of the fluorescence emission 45 released from Rhodamine passes through the filter 52b to be photoelectrically detected by the photomultiplier 50.

As described above, since the optical head 35 is moved on the base plate 63 in the X direction in FIG. 12 by the main scanning pulse motor 65 mounted on the base plate 63 and the base plate 63 is moved in the Y direction in FIG. 12 by the sub-scanning pulse motor 61, all of the absorptive regions 4 formed in the biochemical analysis unit 1 are scanned by the laser beam 24. Therefore, the photomultiplier 50 can read fluorescent data of Rhodamine recorded in the biochemical analysis unit 1 by photoelectrically detecting the fluorescence emission 45 released from Rhodamine contained in a number of the absorptive regions 4 formed in the biochemical analysis unit 1 and produce analog data for biochemical analysis.

The analog data produced by photoelectrically detecting the stimulated emission 45 with the photomultiplier 50 are converted by the A/D converter 53 into digital data and the digital data are fed to the data processing apparatus 54.

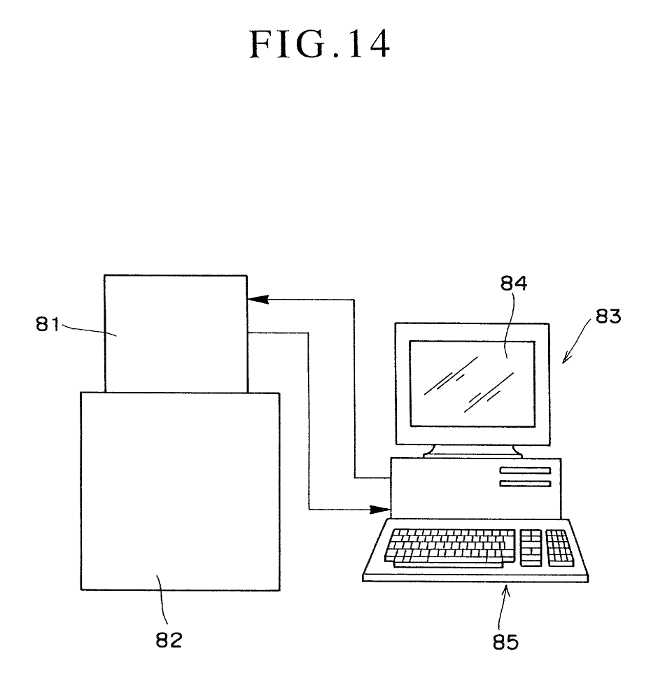

FIG. 14 is a schematic front view showing a data producing system for reading chemiluminescent data of a labeling substance recorded in absorptive regions formed in the biochemical analysis unit 1, which generates chemiluminescent emission when it contacts a chemiluminescent substrate and producing biochemical analysis data.

The data producing system shown in FIG. 14 is constituted to be able to also read fluorescence data of a fluorescent substance such as a fluorescent dye recorded in a number of the absorptive regions 4 in the biochemical analysis unit 1.

As shown in FIG. 14, the data producing system includes a cooled CCD camera 81, a dark box 82 and a personal computer 83. As shown in FIG. 14, the personal computer 83 is equipped with a CRT 84 and a keyboard 85.

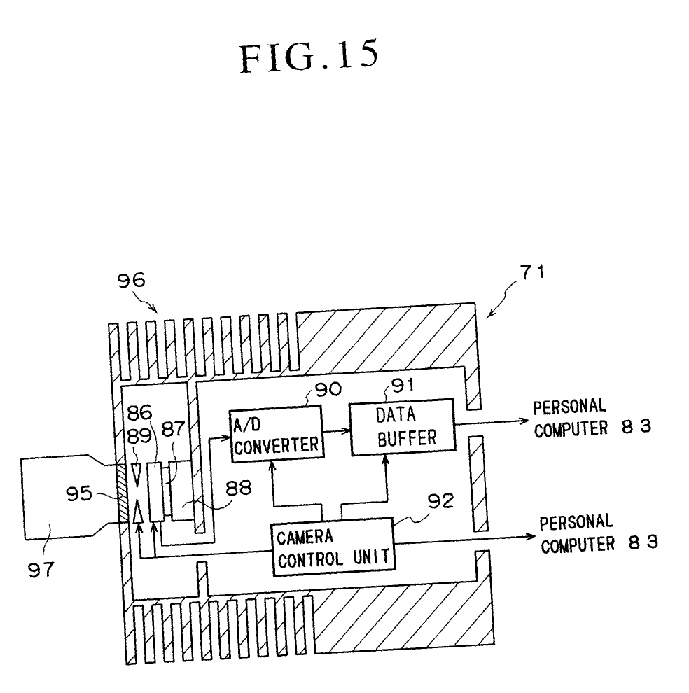

FIG. 15 is a schematic longitudinal cross sectional view showing the cooled CCD camera 81.

As shown in FIG. 15, the cooled CCD camera 81 includes a CCD 86, a heat transfer plate 87 made of metal such as aluminum, a Peltier element 88 for cooling the CCD 86, a shutter 89 disposed in front of the CCD 86, an A/D converter 90 for converting analog data produced by the CCD 86 to digital data, a data buffer 91 for temporarily storing the data digitized by the A/D converter 90, and a camera control circuit 92 for controlling the operation of the cooled CCD camera 81. An opening formed between the dark box 82 and the cooled CCD camera 81 is closed by a glass plate 95 and the periphery of the cooled CCD camera 81 is formed with heat dispersion fins 96 over substantially its entire length for dispersing heat.

A camera lens 97 disposed in the dark box 82 is mounted on the front surface of the glass plate 95 disposed in the cooled CCD camera 81.

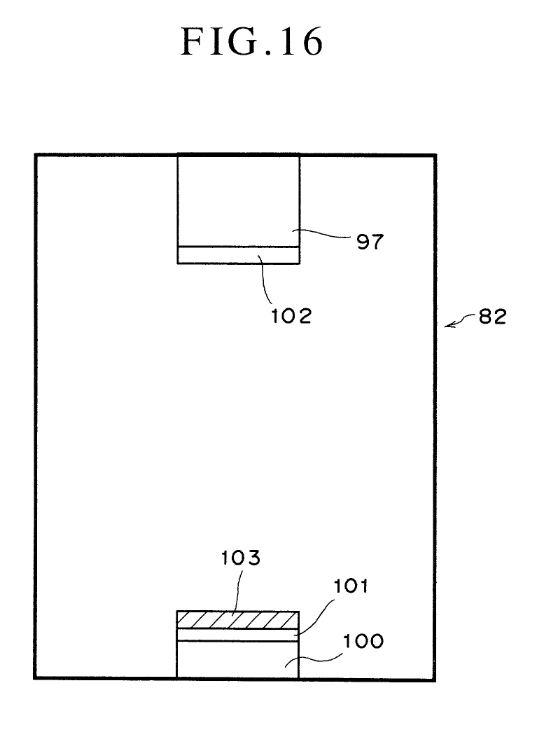

FIG. 16 is a schematic vertical cross sectional view showing the dark box 82.

As shown in FIG. 16, the dark box 82 is equipped with a light emitting diode stimulating ray source 100 for emitting a stimulating ray. The light emitting diode stimulating ray source 100 is provided with a filter 101 detachably mounted thereon and a diffusion plate 102 mounted on the upper surface of the filter 101. The stimulating ray is emitted via the diffusion plate 102 toward a biochemical analysis unit (not shown) placed on the diffusion plate 102 so as to ensure that the biochemical analysis unit can be uniformly irradiated with the stimulating ray. The filter 101 has a property of cutting light components having a wavelength not close to that of the stimulating ray and harmful to the stimulation of a fluorescent substance and transmitting through only light components having a wavelength in the vicinity of that of the stimulating ray. A filter 102 for cutting light components having a wavelength in the vicinity of that of the stimulating ray is detachably provided on the front surface of the camera lens 97.

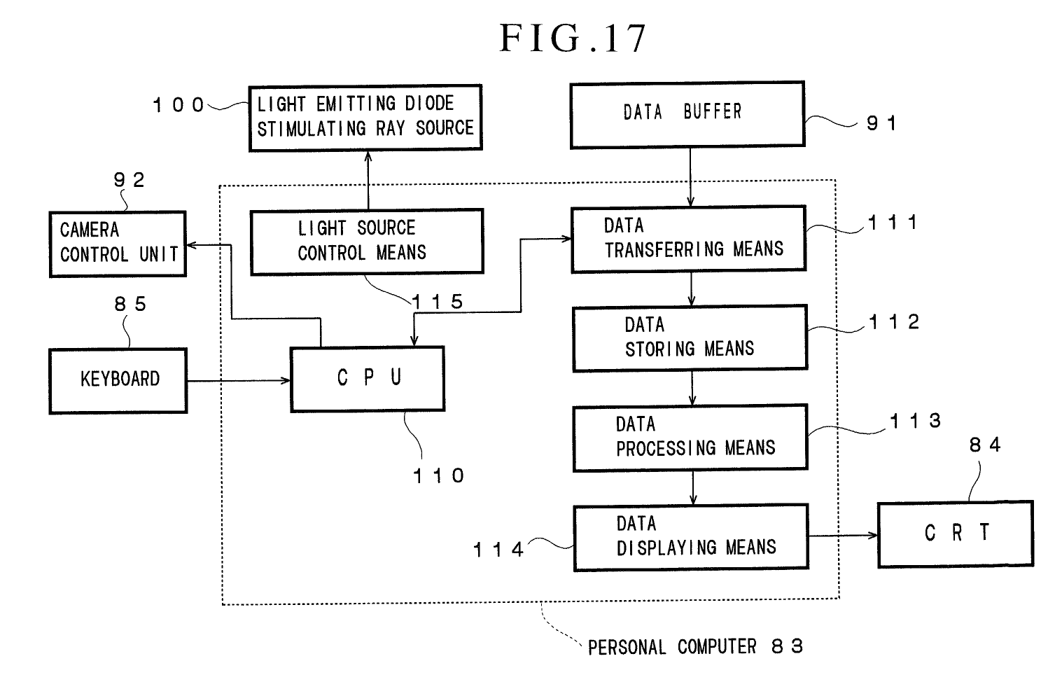

FIG. 17 is a block diagram of the personal computer 83 and peripheral devices thereof.

As shown in FIG. 17, the personal computer 83 includes a CPU 110 for controlling the exposure of the cooled CCD camera 81, a data transferring means 111 for reading the data produced by the cooled CCD camera 81 from the data buffer 91, a storing means 112 for storing data, a data processing means 113 for effecting data processing on the digital data stored in the data storing means 112, and a data displaying means 114 for displaying visual data on the screen of the CRT 84 based on the digital data stored in the data storing means 112. The light emitting diode stimulating ray source 100 is controlled by a light source control means 115 and an instruction signal can be input via the CPU 110 to the light source control means 115 through the keyboard 85. The CPU 110 is constituted so as to output various signals to the camera controlling circuit 93 of the cooled CCD camera 81.

The data producing system shown in FIGS. 14 to 17 is constituted so as to detect chemiluminescent emission generated by the contact of a labeling substance contained in a number of the absorptive regions 4 formed in the biochemical analysis unit 1 and a chemiluminescent substrate, with the CCD 86 of the cooled CCD camera 81 through a camera lens 97, thereby reading chemiluminescence data to produce biochemical analysis data, and irradiate the biochemical analysis unit 1 with a stimulating ray emitted from the light emitting diode stimulating ray source 100 and detect fluorescence emission released from a fluorescent substance such as a fluorescent dye contained in a number of the absorptive regions 4 formed in the biochemical analysis unit 1 upon being stimulated, with the CCD 86 of the cooled CCD camera 81 through a camera lens 97, thereby reading fluorescence data to produce biochemical analysis data.

When biochemical analysis data are to be produced by reading chemiluminescence data, the filter 102 is removed and while the light emitting diode stimulating ray source 100 is kept off, the biochemical analysis unit 1 is placed on the diffusion plate 103, which is releasing chemiluminescent emission as a result of contact of a labeling substance contained in a number of the absorptive regions 4 formed in the biochemical analysis unit 1 and a chemiluminescent substrate.

The lens focus is then adjusted by the user using the camera lens 97 and the dark box 92 is closed.

When an exposure start signal is input by the user through the keyboard 85, the exposure start signal is input through the CPU 110 to the camera control circuit 92 of the cooled CCD camera 81 so that the shutter 88 is opened by the camera control circuit 92, whereby the exposure of the CCD 86 is started.

Chemiluminescent emission released from a number of the absorptive regions 4 of the biochemical analysis unit 1 impinges on the light receiving surface of the CCD 86 of the cooled CCD camera via the camera lens 97, thereby forming an image on the light receiving surface. The CCD 86 receives light of the thus formed image and accumulates it in the form of electric charges therein.

In this embodiment, since the covering regions 3 of gold are formed on the absorptive substrate 2 and neighboring absorptive regions 4 are isolated by the covering regions 3 of gold, it is possible to reliably prevent chemiluminescent emission released from the labeling substance contained in each of the absorptive regions 4 from being mixed with chemiluminescent emission released from a labeling substance contained in the neighboring absorptive regions 4.

When a predetermined exposure time has passed, the CPU 110 outputs an exposure completion signal to the camera control circuit 92 of the cooled CCD camera 81.

When the camera controlling circuit 92 receives the exposure completion signal from the CPU 110, it transfers analog data accumulated in the CCD 86 in the form of electric charge to the A/D converter 90 to cause the A/D converter 90 to digitize the data and to temporarily store the thus digitized data in the data buffer 91.

At the same time, the CPU 110 outputs a data transfer signal to the data transferring means 111 to cause it to read out the digital data from the data buffer 91 of the cooled CCD camera 81 and to input them to the data storing means 112.

When the user inputs a data producing signal through the keyboard 85, the CPU 110 outputs the digital data stored in the data storing means 112 to the data processing means 113 and causes the data processing means 113 to effect data processing on the digital data in accordance with the user's instructions. The CPU 110 then outputs a data display signal to the displaying means 115 and causes the displaying means 115 to display biochemical analysis data on the screen of the CRT 84 based on the thus processed digital data.

On the other hand, when biochemical analysis data are to be produced by reading fluorescence data, the biochemical analysis unit is first placed on the diffusion plate 103.

The light emitting diode stimulating ray source 100 is then turned on by the user and the lens focus is adjusted using the camera lens 97. The dark box 92 is then closed.

When the user inputs an exposure start signal through the keyboard 85, the light emitting diode stimulating ray source 100 is again turned on by the light source control means 115, thereby emitting a stimulating ray toward the biochemical analysis unit 1.

At the same time, the exposure start signal is input via the CPU 110 to the camera control circuit 92 of the cooled CCD camera and the shutter 89 is opened by the camera control circuit 92, whereby the exposure of the CCD 86 is started.

The stimulating ray emitted from the light emitting diode stimulating ray source 100 passes through the filter 101, whereby light components of wavelengths not in the vicinity of that of the stimulating ray are cut. The stimulating ray then passes through the diffusion plate 103 to be made uniform light and the biochemical analysis unit 1 is irradiated with the uniform stimulating ray.

When the biochemical analysis unit 1 is irradiated with the stimulating ray, a fluorescent substance such as a fluorescent dye contained in a number of the absorptive regions 4 of the biochemical analysis unit 1 is stimulated by the stimulating ray, thereby releasing fluorescence emission from a number of the absorptive regions 4 of the biochemical analysis unit 1.

The fluorescence emission released from a number of the absorptive regions 4 of the biochemical analysis unit 1 impinges on the light receiving surface of the CCD 86 of the cooled CCD camera through the filter 102 and the camera lens 97 and forms an image thereon. The CCD 86 receives light of the thus formed image and accumulates it in the form of electric charges therein. Since light components of wavelength equal to the stimulating ray wavelength are cut by the filter 102, only fluorescence emission released from the fluorescent substance such as a fluorescent dye contained in a number of the absorptive regions 4 of the biochemical analysis unit 1 is received by the CCD 86.

In this embodiment, since the covering regions 3 of gold are formed on the absorptive substrate 2 and neighboring absorptive regions 4 are isolated by the covering regions 3 of gold, it is possible to reliably prevent fluorescence emission released from the fluorescent substance such as a fluorescent dye contained in each of the absorptive regions 4 from being mixed with fluorescence emission released from a fluorescent substance as a fluorescent dye contained in the neighboring absorptive regions 4.

When a predetermined exposure time has passed, the CPU 110 outputs an exposure completion signal to the camera control circuit 92 of the cooled CCD camera 81.

When the camera controlling circuit 92 receives the exposure completion signal from the CPU 110, it transfers analog data accumulated in the CCD 86 in the form of electric charge to the A/D converter 90 to cause the A/D converter 90 to digitize the data and to temporarily store the thus digitized data in the data buffer 91.

At the same time, the CPU 110 outputs a data transfer signal to the data transferring means 111 to cause it to read out the digital data from the data buffer 91 of the cooled CCD camera 81 and to input them to the data storing means 112.

When the user inputs a data producing signal through the keyboard 85, the CPU 110 outputs the digital data stored in the data storing means 112 to the data processing apparatus 113 and causes the data processing apparatus 113 to effect data processing on the digital data in accordance with the user's instructions. The CPU 110 then outputs a data display signal to the displaying means 115 and causes the displaying means 115 to display biochemical analysis data on the screen of the CRT 84 based on the thus processed digital data.

In this embodiment, the biochemical analysis unit 1 includes the absorptive substrate 2 and the covering regions 3 of gold are formed on the absorptive substrate 2, thereby forming a number of substantially circular absorptive regions at which the absorptive substrate 2 is exposed. A solution containing specific binding substances such as a plurality of cDNAs whose sequences are known but are different from each other are spotted into in a number of the absorptive regions 4 formed in the biochemical analysis unit 1 using the spotting device 5 and the specific binding substances are fixed therein.

A hybridization solution 9 containing a substance derived from a living organism labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate is prepared and the biochemical analysis unit 1 is accommodated in the hybridization reaction vessel 8 containing the thus prepared hybridization solution 9, whereby specific binding substances spotted in a number of the absorptive regions 4 of the biochemical analysis unit 1 are hybridized with the substances derived from a living organism contained in the hybridization solution 9 and the specific binding substances are selectively labeled with a radioactive labeling substance, a fluorescent substance such as a fluorescent dye and a labeling substance which generates chemiluminescent emission when it contacts a chemiluminescent substrate, whereby radiation data, fluorescent data and chemiluminescent data are recorded in a number of the absorptive regions 4 of the biochemical analysis unit 1.

When radiation data recorded in a number of the absorptive regions 4 of the biochemical analysis unit 1 are to be transferred to the stimulable phosphor layer 12 of the stimulable phosphor sheet by exposing the stimulable phosphor layer 12 formed on the support 11 of the stimulable phosphor sheet 10 to a radioactive labeling substance, the stimulable phosphor sheet 10 is superposed on the biochemical analysis unit 1 in such a manner that the stimulable phosphor layer 12 uniformly formed on one surface of the support 11 of the stimulable phosphor sheet 10 comes into close contact with the covering regions 3 of gold, thereby exposing the stimulable phosphor layer 12 of the stimulable phosphor sheet 10 to the radioactive labeling substance selectively contained in a number of the absorptive regions 4 of the biochemical analysis unit 1.

Therefore, according to this embodiment, during the exposure operation, although electron beams (β rays) are released from the radioactive labeling substance contained in the absorptive regions 4 of the biochemical analysis unit 1, since the covering regions 3 of gold are formed on the surface of the absorptive substrate 2 and neighboring absorptive regions 4 are isolated by the covering regions of gold, electron beams (β rays) released from the radioactive labeling substance contained in each of the absorptive regions 4 can be effectively prevented from entering regions of the stimulable phosphor layer 12 other than a region the of the absorptive regions 4 faces. Further, since gold is vapor-deposited onto the surface of the absorptive substrate 2 so that the thickness of the covering region 3 of gold is about double the diameter of the individual absorptive regions 4, the electron beams (β rays) released from the radioactive labeling substance contained in each of the absorptive regions 4 are prevented by the collimation effect from broadening. Therefore, since it is possible to selectively expose only the region of the stimulable phosphor layer 12 each of the absorptive regions 4 faces to the electron beams (β rays) released from the radioactive labeling substance contained in each of the absorptive regions 4, even in the case where absorptive regions 4 to which specific binding substances are to be spotted are formed in the biochemical analysis unit 1 at high density, it is possible to effectively prevent noise caused by the scattering of the electron beams (β rays) from being generated in biochemical analysis data produced by exciting stimulable phosphor contained in the stimulable phosphor layer 12 with a laser beam 24 and photoelectrically detecting stimulated emission released from the stimulable phosphor and, therefore, to produce biochemical analysis data having high quantitative accuracy.

Further, according to the above described embodiment, since the covering regions 3 of gold are formed on the surface of the absorptive substrate 2 and neighboring absorptive regions 4 are isolated by the covering regions 3 of gold, when each of the absorptive regions 4 of the biochemical analysis unit 1 is irradiated with a laser beam 24 or a stimulating ray emitted from the stimulating ray source 100, the laser beam 24 or the stimulating ray can be prevented from scattering and stimulating a fluorescent substance such as a fluorescent dye contained in the neighboring absorptive regions 4. Therefore, even in the case where absorptive regions 4 to which specific binding substances are to be spotted are formed in the biochemical analysis unit 1 at high density, it is possible to effectively prevent noise caused by the scattering of the laser beam 24 or the stimulating ray from being generated in biochemical analysis data produced by exciting the fluorescent substance such as the fluorescent dye contained in a number of absorptive regions 4 and photoelectrically detecting fluorescence emission released from a number of absorptive regions 4 and, therefore, to produce biochemical analysis data having high quantitative accuracy.

Furthermore, according to the above described embodiment, since the covering regions 3 of gold are formed on the surface of the absorptive substrate 2 and neighboring absorptive regions 4 are isolated by the covering regions 3 of gold, fluorescence emission or chemiluminescent emission released from each of the absorptive regions 4 of the biochemical analysis unit 1 can be effectively prevented from being mixed with fluorescence emission or chemiluminescent emission released from neighboring absorptive regions 4. Therefore, even in the case where absorptive regions 4 to which specific binding substances are to be spotted are formed in the biochemical analysis unit 1 at high density, it is possible to effectively prevent noise caused by the scattering of the laser beam or the stimulating ray from being generated in biochemical analysis data produced by photoelectrically detecting fluorescence emission or chemiluminescent emission released from a number of absorptive regions 4 and, therefore, to produce biochemical analysis data having high quantitative accuracy.

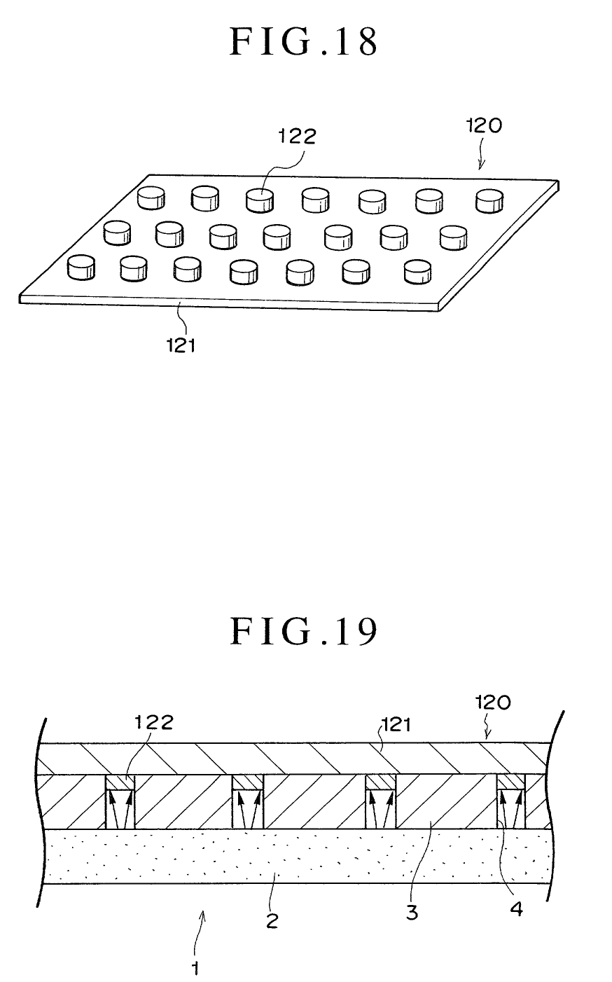

FIG. 18 is a schematic perspective view showing a stimulable phosphor sheet used in a biochemical analyzing method according to another preferred embodiment of the present invention.

As shown in FIG. 18, a stimulable phosphor sheet 120 according to this embodiment includes a support 121 and a number of stimulable phosphor layer regions 122 are formed on one surface of the support 121 in the same pattern as that of the absorptive regions 4 formed in the biochemical analysis unit 1 so that each of them has substantially the same size as that of the individual absorptive regions 4.

Therefore, although not accurately shown in FIG. 18, in this embodiment, the stimulable phosphor layer regions 122 having a size of about 0.07 cm² are regularly formed on the support 121 of the stimulable phosphor sheet 120 in a matrix manner of 120 columns×160 lines and, therefore, 19,200 stimulable phosphor layer regions are formed.

In this embodiment, the support 121 of the stimulable phosphor sheet 120 is formed of stainless steel capable of attenuating radiation energy.

Similarly to the embodiment shown in FIGS. 1 to 17, in this embodiment, a solution containing specific binding substances such as cDNAs are spotted onto a number of the absorptive regions 4 formed in the biochemical analysis unit 1 using the spotting device 5 shown in FIG. 3 and the specific binding substances contained in a number of the absorptive regions 4 formed in the biochemical analysis unit 1 are selectively hybridized with a substance derived from a living organism and labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate using the hybridization reaction vessel 8 shown in FIG. 4, whereby radiation data, fluorescent data and chemiluminescent data are recorded in a number of the absorptive regions 4 of the biochemical analysis unit 1.

The fluorescent data recorded in a number of the absorptive regions 4 of the biochemical analysis unit 1 in this manner are read by the scanner shown in FIGS. 6 to 14 similarly to the previous embodiment and biochemical analysis data are produced.

On the other hand, the chemiluminescent data recorded in a number of the absorptive regions 4 of the biochemical analysis unit 1 in this manner are read by the data producing system shown in FIGS. 14 to 17 similarly to the previous embodiment and biochemical analysis data are produced.

To the contrary, the radiation data recorded in a number of the absorptive regions 4 of the biochemical analysis unit 1 in this manner are transferred into a number of stimulable phosphor layer regions 122 formed on the support 121 of the stimulable phosphor sheet 120.

FIG. 19 is a schematic cross-sectional view showing a method for exposing a number of the stimulable phosphor layer regions 122 formed on the support 121 of the stimulable phosphor sheet 120 by a radioactive labeling substance selectively contained in a number of the absorptive regions 4 of the biochemical analysis unit 1.

As shown in FIG. 19, when stimulable phosphor contained in the stimulable phosphor layer regions 122 is to be exposed to a radioactive labeling substance, the stimulable phosphor sheet 120 is superposed on the biochemical analysis unit 1 in such a manner that each of the stimulable phosphor layer regions 122 formed on the support 121 of the stimulable phosphor sheet 120 comes into close contact with the surface of the corresponding absorptive region 4 formed in the biochemical analysis unit 1 and that the periphery of each of the stimulable phosphor layer regions 122 is surrounded by the covering regions 3 of gold.

In this embodiment, since the covering regions 3 of gold are formed on the surface of the absorptive substrate 2 of the biochemical analysis unit 1, the biochemical analysis unit is hardly stretched and shrunk even when it is subjected to liquid processing such as hybridization and, therefore, it is possible to easily and accurately superpose the stimulable phosphor sheet 10 on the biochemical analysis unit 1 so that each of the stimulable phosphor layer regions 122 formed on the support 121 of the stimulable phosphor sheet 120 comes into close contact with the surface of corresponding absorptive region 4 formed in the biochemical analysis unit 1 and that the periphery of each of the stimulable phosphor layer regions 122 is surrounded by the covering regions 3 of gold, thereby exposing stimulable phosphor contained in the stimulable phosphor layer regions 122 to a radioactive labeling substance.

In this manner, the surface of each of the stimulable phosphor layer regions 122 of the stimulable phosphor sheet 120 is kept in close contact with the surface of the corresponding absorptive region of the biochemical analysis unit 1 for a predetermined time period, whereby stimulable phosphor contained in the stimulable phosphor layer regions 122 is exposed to a radioactive labeling substance selectively contained in a number of the absorptive regions 4 of the biochemical analysis unit 1.

During the exposure operation, electron beams (β rays) are released from the radioactive labeling substance contained in the absorptive regions 4 of the biochemical analysis unit 1. However, since the covering regions 3 of gold are formed on the surface of the absorptive substrate 2, whereby neighboring absorptive regions 4 are isolated by the covering regions 3 of gold and the periphery of each of the stimulable phosphor layer regions 122 formed on the support 121 of the stimulable phosphor sheet 120 is surrounded by the covering regions 3 of gold, electron beams (β rays) released from the radioactive labeling substance contained in each of the absorptive regions 4 can be effectively prevented from entering stimulable phosphor layer regions 122 other than the stimulable phosphor layer region 122 the of the absorptive regions 4 faces and, therefore, it is possible to selectively expose only the stimulable phosphor layer region 122 each of the absorptive regions 4 faces to the electron beams (β rays) released from the radioactive labeling substance contained in each of the absorptive regions 4 and to reliably prevent the electron beams (β rays) released from the radioactive labeling substance contained in each of the absorptive regions 4 from scattering in the corresponding stimulable phosphor layer region 122 to reach the neighboring stimulable phosphor layer regions 122.

Further, in this embodiment, since gold is vapor-deposited onto the surface of the absorptive substrate 2 so that the thickness of the covering region 3 of gold is about double the diameter of the individual absorptive regions 4, the electron beams (β rays) released from the radioactive labeling substance contained in each of the absorptive regions 4 are prevented by the collimation effect from broadening. Further, since the support 121 of the stimulable phosphor sheet 120 is formed of stainless steel capable of attenuating radiation energy, it is also possible to reliably prevent the electron beams (β rays) from scattering in the support 121 of the stimulable phosphor sheet 120 and entering the neighboring stimulable phosphor layer regions 122 to excite stimulable phosphor contained in the neighboring stimulable phosphor layer regions 122.

In this manner, radiation data are recorded in a number of the stimulable phosphor layer regions 122 of the stimulable phosphor sheet 120 and the radiation data recorded in a number of the stimulable phosphor layer regions 122 of the stimulable phosphor sheet 120 are read by the scanner shown in FIGS. 6 to 13 similarly to the previous embodiment and biochemical analysis data are produced.

According to this embodiment, since stimulable phosphor contained in each of the stimulable phosphor layer regions 122 formed on the support 121 of the stimulable phosphor sheet 120 can be selectively exposed to only the electron beams (β rays) released from a radioactive labeling substance contained in an absorptive regions 4 of the biochemical analysis unit 1 the stimulable phosphor layer region 122 faces, even in the case where absorptive regions 4 to which specific binding substances are to be spotted are formed in the biochemical analysis unit 1 at high density, it is possible to effectively prevent noise caused by the scattering of the electron beams (β rays) from being generated in biochemical analysis data produced by exciting stimulable phosphor contained in the stimulable phosphor layer 12 with a laser beam 24 and photoelectrically detecting stimulated emission released from the stimulable phosphor and, therefore, to produce biochemical analysis data having high quantitative accuracy.

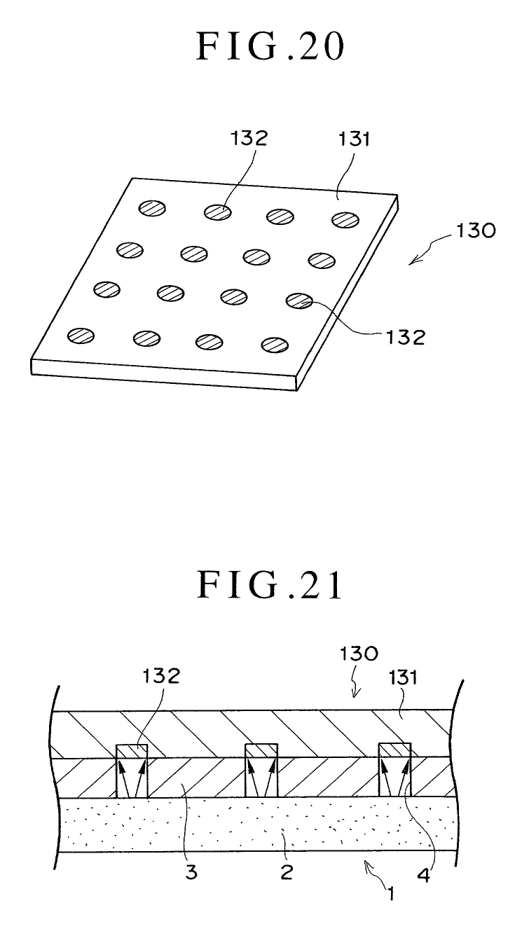

FIG. 20 is a schematic perspective view showing a stimulable phosphor sheet used in a biochemical analyzing method according to a further preferred embodiment of the present invention.

As shown in FIG. 20, a stimulable phosphor sheet 130 according to this embodiment includes a support 131 formed with a number of recesses having substantially the same size as that of the absorptive regions 4 in the same pattern as that of a number of the absorptive regions 4 formed in the biochemical analysis unit 1 on one side surface thereof and a number of stimulable phosphor layer regions 132 are formed by embedding stimulable phosphor into a number of the recesses formed on the one side surface of the support 131 so that the pattern thereof is the same as that of the absorptive regions 4 formed in the biochemical analysis unit 1 and the size thereof is substantially the same as that of the absorptive region 4.

In this embodiment, a number of the stimulable phosphor layer regions 132 are formed by embedding stimulable phosphor into the recesses so that the surface of each of the stimulable phosphor layer regions 132 is located at the same level as that of the surface of the support 131.

Therefore, although not accurately shown in FIG. 20, in this embodiment, the stimulable phosphor layer regions 132 having a size of about 0.07 cm² are regularly formed in the support 131 of the stimulable phosphor sheet 130 in a matrix manner of 120 columns×160 lines and, therefore, 19,200 stimulable phosphor layer regions are formed.

In this embodiment, the support 131 of the stimulable phosphor sheet 130 is formed of stainless steel capable of attenuating radiation energy.

Similarly to the embodiment shown in FIGS. 1 to 17, in this embodiment, a solution containing specific binding substances such as cDNAs are spotted onto a number of the absorptive regions 4 formed in the biochemical analysis unit 1 using the spotting device 5 shown in FIG. 3 and the specific binding substances contained in a number of the absorptive regions 4 formed in the biochemical analysis unit 1 are selectively hybridized with a substance derived from a living organism and labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate using the hybridization reaction vessel 8 shown in FIG. 4, whereby radiation data, fluorescent data and chemiluminescent data are recorded in a number of the absorptive regions 4 of the biochemical analysis unit 1.

The fluorescent data recorded in a number of the absorptive regions 4 of the biochemical analysis unit 1 in this manner are read by the scanner shown in FIGS. 6 to 14 similarly to the first mentioned embodiment and biochemical analysis data are produced.

On the other hand, the chemiluminescent data recorded in a number of the absorptive regions 4 of the biochemical analysis unit 1 in this manner are read by the data producing system shown in FIGS. 14 to 17 similarly to the first mentioned embodiment and biochemical analysis data are produced.

To the contrary, the radiation data recorded in a number of the absorptive regions 4 of the biochemical analysis unit 1 in this manner are transferred into a number of stimulable phosphor layer regions 132 formed in the support 131 of the stimulable phosphor sheet 130.

FIG. 21 is a schematic cross-sectional view showing a method for exposing a number of the stimulable phosphor layer regions 132 formed in the stimulable phosphor sheet 130 by a radioactive labeling substance selectively contained in a number of the absorptive regions 4 of the biochemical analysis unit 1.

As shown in FIG. 21, when stimulable phosphor contained in the stimulable phosphor layer regions 122 is to be exposed to a radioactive labeling substance, the stimulable phosphor sheet 130 is superposed on the biochemical analysis unit 1 in such a manner that the surface of the support 121 of the stimulable phosphor sheet 120 comes into close contact with the surface of the covering regions of gold formed on the surface of the absorptive substrate 2 of the biochemical analysis unit 1, whereby each of the stimulable phosphor layer regions 132 formed in the support 131 of the stimulable phosphor sheet 130 faces corresponding absorptive region of the biochemical analysis unit 1.

During the exposure operation, electron beams (β rays) are released from the radioactive labeling substance contained in the absorptive regions 4 of the biochemical analysis unit 1. However, since the covering regions 3 of gold are formed on the surface of the absorptive substrate 2 and neighboring absorptive regions 4 are isolated by the covering regions 3 of gold, the electron beams (β rays) released from the radioactive labeling substance contained in each of the absorptive regions 4 can be effectively prevented from entering stimulable phosphor layer regions 132 other than the stimulable phosphor layer region 132 each of the absorptive regions 4 faces and, therefore, it is possible to selectively expose only the stimulable phosphor layer region 122 each of the absorptive regions 4 faces to the electron beams (β rays) released from the radioactive labeling substance contained in each of the absorptive regions 4.

In this manner, radiation data are recorded in a number of the stimulable phosphor layer regions 132 of the stimulable phosphor sheet 130 and the radiation data recorded in a number of the stimulable phosphor layer regions 132 of the stimulable phosphor sheet 130 are read by the scanner shown in FIGS. 6 to 13 similarly to the first mentioned embodiment and biochemical analysis data are produced.

According to this embodiment, since stimulable phosphor contained in each of the stimulable phosphor layer regions 132 formed on the support 131 of the stimulable phosphor sheet 130 can be selectively exposed to only the electron beams (β rays) released from a radioactive labeling substance contained in an absorptive regions 4 of the biochemical analysis unit 1 the stimulable phosphor layer region 122 faces, even in the case where absorptive regions 4 to which specific binding substances are to be spotted are formed in the biochemical analysis unit 1 at high density, it is possible to effectively prevent noise caused by the scattering of the electron beams (β rays) from being generated in biochemical analysis data produced by exciting stimulable phosphor contained in the stimulable phosphor layer 12 with a laser beam 24 and photoelectrically detecting stimulated emission released from the stimulable phosphor and, therefore, to produce biochemical analysis data having high quantitative accuracy.

The present invention has thus been shown and described with reference to specific embodiments. However, it should be noted that the present invention is in no way limited to the details of the described arrangements but changes and modifications may be made without departing from the scope of the appended claims.

For example, in the above-described embodiments, as specific binding substances, cDNAs each of which has a known base sequence and is different from the others are used. However, specific binding substances usable in the present invention are not limited to cDNAs but all specific binding substances capable of specifically binding with a substance derived from a living organism such as a hormone, tumor marker, enzyme, antibody, antigen, abzyme, other protein, a nuclear acid, cDNA, DNA, RNA or the like and whose sequence, base length, composition and the like are known, can be employed in the present invention as a specific binding substance.

Further, in the above-described embodiments, although the covering regions 3 of gold are formed on the surface of the absorptive substrate 3 of the biochemical analysis unit 1, it is not absolutely necessary to form the covering regions 5 of gold but the covering regions 5 may be formed of silver, copper, zinc, aluminum, titanium, tantalum, chromium, iron, nickel, cobalt, lead, tin or the like or alloys thereof instead of gold.

Furthermore, in the above-described embodiments, although the covering regions 3 of gold are formed by vapor depositing gold onto the surface of the absorptive substrate 3 of the biochemical analysis unit 1, it is not absolutely necessary to form the covering regions 5 by vapor deposition but covering regions 3 may be formed by means of sputtering, chemical vapor deposition or the like instead of vapor deposition of gold.

Moreover, in the above described embodiments, gold is vapor-deposited onto the surface of the absorptive substrate 2 of the biochemical analysis unit 1 so that the thickness of the covering layer 5 of gold is about double the diameter of each of the absorptive regions 4. However, it is not absolutely necessary to vapor deposit gold onto the surface of the absorptive substrate 2 of the biochemical analysis unit 1 so that the thickness of the covering layer 5 of gold is about double the diameter of each of the absorptive regions 4 and the thickness of the covering layer 5 of gold and the diameter or breadth of each of the absorptive regions 4 can be arbitrarily determined. The covering layer 5 of gold is preferably formed to have a thickness of 0.5 to 100 times the diameter or the maximum breadth of the individual absorptive regions and more preferably 1 to 10 times the diameter of the individual absorptive regions.

Further, in the above described embodiments, although 19,200 of substantially circular absorptive regions 4 having a size of about 0.07 cm² are regularly formed in the biochemical analysis unit in a matrix manner of 120 columns×160 lines, the number or size of the absorptive regions 4 may be arbitrarily selected in accordance with the purpose. Preferably, 10 or more of the absorptive regions 4 having a size of 5 cm² or less are formed in the biochemical analysis unit 1 at a density of 10/cm² or less.

Furthermore, in the above described embodiments, although 19,200 of substantially circular absorptive regions 4 having a size of about 0.07 cm² are regularly formed in the biochemical analysis unit in a matrix manner of 120 columns×160 lines, it is not absolutely necessary to regularly form the absorptive regions 4 in the biochemical analysis unit 1.

Moreover, in the above described embodiments, although each of the absorptive regions 4 are formed substantially circular, the shape of each of the absorptive regions 4 is not limited to substantially a circular shape and may be arbitrarily selected.

Further, in the above-described embodiments, a hybridization solution 9 containing a substance derived from a living organism labeled with a radioactive labeling substance, a substance derived from a living organism labeled with a fluorescent substance such as a fluorescent dye and a substance derived from a living organism labeled with a labeling substance which generates chemiluminescent emission when it contacts a chemiluminescent substrate is prepared and the substance derived from a living organism is selectively hybridized with the specific binding substances contained in a number of the absorptive regions of the biochemical analysis unit 1. However, it is not absolutely necessary for substances derived from a living organism contained in a hybridization solution 9 to be labeled with a radioactive labeling substance, a fluorescent substance and a labeling substance which generates chemiluminescent emission when it contacts a chemiluminescent substrate and it is sufficient for substances derived from a living organism contained in a hybridization solution 9 to be labeled with at least one kind of a labeling substance selected from a group consisting of a radioactive labeling substance, a fluorescent substance and a labeling substance which generates chemiluminescent emission when it contacts a chemiluminescent substrate.

Furthermore, in the above-described embodiments, specific binding substances are hybridized with substances derived from a living organism labeled with a radioactive labeling substance, a fluorescent substance and a labeling substance which generates chemiluminescent emission when it contacts a chemiluminescent substrate. However, it is not absolutely necessary to hybridize substances derived from a living organism with specific binding substances and substances derived from a living organism may be specifically bound with specific binding substances by means of antigen-antibody reaction, receptor-ligand reaction or the like instead of hybridization.

Moreover, in the embodiment shown in FIGS. 18 and 19 and the embodiment shown in FIGS. 20 and 21, although the support 121 of the stimulable phosphor sheet 120 and the support 131 of the stimulable phosphor sheet 130 are made of stainless steel, it is sufficient for the support 121 and the support 131 to be made of a material capable of attenuating radiation energy and the support 121 and the support 131 can be formed of either inorganic compound material or organic compound material and is preferably formed of metal material, ceramic material or plastic material. Illustrative examples of inorganic compound materials include metals such as gold, silver, copper, zinc, aluminum, titanium, tantalum, chromium, steel, nickel, cobalt, lead, tin, selenium and the like; alloys such as brass, stainless, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds are preferably used as organic compound material and illustrative examples thereof include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4, 10 and the like; polyimide; polysulfone; polyphenylene sulfide; silicon resins such as polydiphenyl siloxane and the like; phenol resins such as novolac and the like; epoxy resin; polyurethane; polystyrene, butadienestyrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials.

Furthermore, in the above-described embodiments, biochemical analysis data are produced by reading radiation data of a radioactive labeling substance recorded in the stimulable phosphor layer 12 of the stimulable phosphor sheet 10, radiation data of a radioactive labeling substance recorded in a number of the stimulable phosphor layer regions 122 of the stimulable phosphor sheet 120, radiation data of a radioactive labeling substance recorded in a number of the stimulable phosphor layer regions 132 of the stimulable phosphor sheet 130 and fluorescence data of a fluorescent substance such as a fluorescent dye recorded in a number of the absorptive regions 4 of the biochemical analysis unit 1 using the scanner shown in FIGS. 6 to 13. However, it is not absolutely necessary to produce biochemical analysis data by reading radiation data of a radioactive labeling substance and fluorescence data of a fluorescent substance using a single scanner and biochemical analysis data may be produced by reading radiation data of a radioactive labeling substance and fluorescence data of a fluorescent substance using separate scanners.

Moreover, in the above-described embodiments, biochemical analysis data are produced by reading radiation data of a radioactive labeling substance recorded in the stimulable phosphor layer 12 of the stimulable phosphor sheet 10, radiation data of a radioactive labeling substance recorded in a number of the stimulable phosphor layer regions 122 of the stimulable phosphor sheet 120, radiation data of a radioactive labeling substance recorded in a number of the stimulable phosphor layer regions 132 of the stimulable phosphor sheet 130 and fluorescence data of a fluorescent substance such as a fluorescent dye recorded in a number of the absorptive regions 4 of the biochemical analysis unit 1 using the scanner shown in FIGS. to 13. However, it is not absolutely necessary to read radiation data of a radioactive labeling substance using the scanner shown in FIGS. 6 to 13 and any scanner constituted so as to scan and stimulate the stimulable phosphor layer 12 formed on the support 11 of the stimulable phosphor layer sheet 10, a number of the stimulable phosphor layer regions 122 formed on the support 121 of the stimulable phosphor sheet 120 and a number of the stimulable phosphor layer regions 132 formed in the support 131 of the stimulable phosphor sheet 130 with a laser beam 24 may be used for reading radiation data of a radioactive labeling substance.

Further, in the above-described embodiments, although the scanner shown in FIGS. 6 to 13 includes the first laser stimulating ray source 1, the second laser stimulating ray source 2 and the third laser stimulating ray source 3, it is not absolutely necessary for the scanner to include three laser stimulating ray sources.

Furthermore, in the above-described embodiments, the data producing system shown in FIGS. 14 to 17 is constituted so as to photoelectrically detect fluorescence emission and chemiluminescent emission and read fluorescent data and chemiluminescent data. However, it is not absolutely necessary to produce biochemical analysis data by reading chemiluminescence data using the data producing system which can also read fluorescence data and in the case where only chemiluminescence data of a labeling substance which generates chemiluminescent emission when it contacts a chemiluminescent substrate recorded in a number of the absorptive regions 4 of the biochemical analysis unit 1 are read, the light emitting diode stimulating ray source 100, the filter 101, the filter 102 and the diffusion plate 102 can be omitted from the data producing system.

Further, in the above described embodiments, chemiluminescent data recorded in a number of the absorptive regions 4 of the biochemical analysis unit 1 are read by the data producing system shown in FIGS. 14 to 17 to produce biochemical analysis data. However, it is possible to read chemiluminescent data by recording chemiluminescent data recorded in a number of the absorptive regions 4 of the biochemical analysis unit 1, causing a number of the absorptive regions 4 of the biochemical analysis unit 1 to come into contact with a chemiluminescent substrate, thereby causing a number of the absorptive regions 4 of the biochemical analysis unit 1 to release chemiluminescent emission, superposing the stimulable phosphor sheet 10 formed with the stimulable phosphor layer 12, the stimulable phosphor sheet 120 formed with a number of the stimulable phosphor regions 122 or the stimulable phosphor layer 12, the stimulable phosphor sheet 130 formed with a number of the stimulable phosphor regions 132 on the biochemical analysis unit 1 whose absorptive regions 4 are releasing chemiluminescent emission, exposing the stimulable phosphor layer of the stimulable phosphor sheet 10, a number of the stimulable phosphor regions 122 of the stimulable phosphor sheet 120 or a number of the stimulable phosphor regions 132 of the stimulable phosphor sheet 130 to chemiluminescent emission released from the absorptive regions 4 of the biochemical analysis unit 1, thereby storing the energy of chemiluminescent emission, scanning the stimulable phosphor layer 12 of the stimulable phosphor sheet 10, a number of the stimulable phosphor regions 122 of the stimulable phosphor sheet 120 or a number of the stimulable phosphor regions 132 of the stimulable phosphor sheet 130 with the laser beam 24 using the scanner shown in FIGS. 6 to 13 similarly to the case of reading radiation data recorded in the stimulable phosphor layer 12 of the stimulable phosphor sheet 10, a number of the stimulable phosphor regions 122 of the stimulable phosphor sheet 120 or a number of the stimulable phosphor regions 132 of the stimulable phosphor sheet 130, and photoelectrically detecting stimulated emission 45 released from the stimulable phosphor layer 12 of the stimulable phosphor sheet 10, a number of the stimulable phosphor regions 122 of the stimulable phosphor sheet 120 or a number of the stimulable phosphor regions 132 of the stimulable phosphor sheet 130 by the photomultiplier 50 and to produce biochemical analysis data.

Moreover, in the above-described embodiments, the scanner shown in FIGS. 6 to 13 is constituted so that the whole surface of the stimulable phosphor layer 12 formed on the support 11 of the stimulable phosphor sheet 10, all of the stimulable phosphor layer regions 122 formed on the support 121 of the stimulable phosphor sheet 120, all of the stimulable phosphor layer regions 132 formed in the support 131 of the stimulable phosphor sheet 130 or all of the absorptive regions 4 of the biochemical analysis unit 1 is scanned with a laser beam 24 to excite stimulable phosphor or a fluorescent substance such as a fluorescent dye by moving the optical head 35 using a scanning mechanism in the X direction and the Y direction in FIG. 12. However, the whole surface of the stimulable phosphor layer 12 formed on the support 11 of the stimulable phosphor sheet 10, all of the stimulable phosphor layer regions 122 formed on the support 121 of the stimulable phosphor sheet 120, all of the stimulable phosphor layer regions 132 formed in the support 131 of the stimulable phosphor sheet 130 or all of the absorptive regions 4 of the biochemical analysis unit 1 can be scanned with a laser beam to excite stimulable phosphor or a fluorescent substance such as a fluorescent dye by moving the stage 40 in the X direction and the Y direction in FIG. 12, while holding the stage 40 stationary. Further, the optical head 35 may be moved in one of the X direction and the Y direction in FIG. 12, while the stage 40 is moved in the other direction.

Furthermore, in the above-described embodiments, although the scanner shown in FIGS. 6 to 13 employs the perforated mirror formed with the hole 33, the mirror can be formed with a coating capable of transmitting the laser beam 24 instead of the hole 33.

Moreover, in the above-described embodiments, the scanner shown in FIGS. 6 to 13 employs the photomultiplier 50 as a light detector to photoelectrically detect fluorescent light or stimulated. However, it is sufficient for the light detector used in the present invention to be able to photoelectrically detect fluorescent light or stimulated emission and it is possible to employ a light detector such as a line CCD or a two-dimensional CCD instead of the photomultiplier 50.

Further, in the above-described embodiments, a solution containing specific binding substances such as cDNAs are spotted using the spotting device 5 including an injector 6 and a CCD camera 7 so that when the tip end portion of the injector 6 and the center of the absorptive region 4 into which a solution containing specific binding substances is to be spotted are determined to coincide with each other as a result of viewing them using the CCD camera 7, the specific binding substance such as cDNA is spotted from the injector 6. However, a solution containing specific binding substances such as cDNAs can be spotted by detecting the positional relationship between a number of the absorptive regions 4 formed in the biochemical analysis unit 1 and the tip end portion of the injector 6 in advance and two-dimensionally moving the biochemical analysis unit 1 or the tip end portion of the injector 6 so that the tip end portion of the injector 6 coincides with each of the absorptive regions 4.

Furthermore, in the above described embodiments, although chemiluminescent emission released from a number of the absorptive regions 4 formed in the biochemical analysis unit 1 is photoelectrically detected by the CCD 86 of the cooled CCD camera to produce biochemical analysis data, biochemical analysis data may be recorded in a recording material such as a photographic film by, for example, exposing the photographic film to chemiluminescent emission released from a number of the absorptive regions 4 formed in the biochemical analysis unit 1.

According to the present invention, it is possible to provide a biochemical analysis unit which can prevent noise caused by the scattering of electron beams released from a radioactive labeling substance from being generated in biochemical analysis data even in the case of forming spots of specific binding substances on the surface of a carrier at high density, which can specifically bind with a substance derived from a living organism and whose sequence, base length, composition and the like are known, specifically binding the spot-like specific binding substance with a substance derived from a living organism and labeled with a radioactive substance to selectively label the spot-like specific binding substances with a radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

Further, according to the present invention, it is possible to provide a biochemical analysis unit which can prevent noise caused by the scattering of chemiluminescent emission and/or fluorescence emission released from a labeling substance which generates chemiluminescent emission when it contacts a chemiluminescent substrate and/or a fluorescent substance from being generated in biochemical analysis data even in the case of forming spots of specific binding substances on the surface of a carrier at high density, which can specifically bind with a substance derived from a living organism and whose sequence, base length, composition and the like are known, specifically binding the spot-like specific binding substance with a substance derived from a living organism and labeled with, in addition to a radioactive labeling substance or instead of a radioactive labeling substance, a labeling substance which generates chemiluminescent emission when it contacts a chemiluminescent substrate and/or a fluorescent substance to selectively label the spot-like specific binding substances therewith, thereby obtaining a biochemical analysis unit, photoelectrically detecting chemiluminescent emission and/or fluorescence emission released from the biochemical analysis unit to produce biochemical analysis data, and analyzing the substance derived from a living organism.

Furthermore, according to the present invention, it is possible to provide a biochemical analyzing method which can effect quantitative biochemical analysis with high accuracy by producing biochemical analysis data based on a biochemical analysis unit obtained by forming spots of specific binding substances on the surface of a carrier at high density, which can specifically bind with a substance derived from a living organism and whose sequence, base length, composition and the like are known, specifically binding the spot-like specific binding substances with a substance derived from a living organism and labeled with a radioactive labeling substance, a labeling substance which generates chemiluminescent emission when it contacts a chemiluminescent substrate and/or a fluorescent substance, thereby selectively labeling the spot-like specific binding substances therewith.

1. A biochemical analysis unit comprising a plurality of absorptive regions formed spaced apart from each other by covering a surface of an absorptive substrate made of an absorptive material with a material capable of attenuating radiation energy and/or light energy.

2. A biochemical analysis unit comprising a plurality of absorptive regions formed spaced apart from each other by covering a surface of an absorptive substrate made of an absorptive material with a material capable of attenuating radiation energy and/or light energy, the plurality of absorptive regions being selectively labeled with at least one kind of labeling substance selected from a group consisting of a radioactive labeling substance, a labeling substance which generates chemiluminescent emission when it contacts a chemiluminescent substrate and a fluorescent substance by spotting specific binding substances whose sequence, base length, composition and the like are known therein and specifically binding a substance derived from a living organism and labeled with at least one kind of said labeling substance with the specific binding substances.

3. A biochemical analysis unit in accordance with claim 2 wherein the substance derived from a living organism is specifically bound with specific binding substances by a reaction selected from a group consisting of hybridization, antigen-antibody reaction and receptor-ligand reaction.

4. A biochemical analysis unit in accordance with claim 1 wherein the material capable of attenuating radiation energy and/or light energy has a property of reducing the energy of radiation and/or light to ⅕ or less when the radiation and/or light travels in the material by a distance equal to that between neighboring absorptive regions.

5. A biochemical analysis unit in accordance with claim 2 wherein the material capable of attenuating radiation energy and/or light energy has a property of reducing the energy of radiation and/or light to ⅕ or less when the radiation and/or light travels in the material by a distance equal to that between neighboring absorptive regions.

6. A biochemical analysis unit in accordance with claim 4 wherein the surface of the absorptive substrate is covered with metal, thereby forming a covering.

7. A biochemical analysis unit in accordance with claim 5 wherein the surface of the absorptive substrate is covered with metal, thereby forming a covering.

8. A biochemical analysis unit in accordance with claim 6 wherein the surface of the absorptive substrate is covered with metal or alloy selected from a group consisting of gold, silver, copper, zinc, aluminum, titanium, tantalum, chromium, iron, nickel, cobalt, lead and tin and alloys thereof, thereby forming a covering.

9. A biochemical analysis unit in accordance with claim 7 wherein the surface of the absorptive substrate is covered with metal or alloy selected from a group consisting of gold, silver, copper, zinc, aluminum, titanium, tantalum, chromium, iron, nickel, cobalt, lead and tin and alloys thereof, thereby forming a covering.

10. A biochemical analysis unit in accordance with claim 1 wherein the absorptive substrate is formed of a porous material or a fiber material.

11. A biochemical analysis unit in accordance with claim 2 wherein the absorptive substrate is formed of a porous material or a fiber material.

12. A biochemical analysis unit in accordance with claim 1 wherein the covering of the material capable of attenuating radiation energy and/or light energy has a thickness of 0.5 to 100 times of the maximum breadth of the individual absorptive regions.

13. A biochemical analysis unit in accordance with claim 2 wherein the covering of the material capable of attenuating radiation energy and/or light energy has a thickness of 0.5 to 100 times of the maximum breadth of the individual absorptive regions.

14. A biochemical analysis unit in accordance with claim 1 wherein the covering of the material capable of attenuating radiation energy and/or light energy has a thickness of 1 to 10 times the maximum breadth of the individual absorptive regions.

15. A biochemical analysis unit in accordance with claim 2 wherein the covering of the material capable of attenuating radiation energy and/or light energy has a thickness of 1 to 10 times the maximum breadth of the individual absorptive regions.

16. A biochemical analysis unit in accordance with claim 1 wherein the biochemical analysis unit is formed with 10 or more absorptive regions.

17. A biochemical analysis unit in accordance with claim 2 wherein the biochemical analysis unit is formed with 10 or more absorptive regions.

18. A biochemical analysis unit in accordance with claim 1 wherein each of the plurality of absorptive regions formed in the biochemical analysis unit has a size of less than 5 mm².

19. A biochemical analysis unit in accordance with claim 2 wherein each of the plurality of absorptive regions formed in the biochemical analysis unit has a size of less than 5 mm².

20. A biochemical analysis unit in accordance with claim 1 wherein the plurality of absorptive regions are formed in the biochemical analysis unit at a density of 10 or more per cm².

21. A biochemical analysis unit in accordance with claim 2 wherein the plurality of absorptive regions are formed in the biochemical analysis unit at a density of 10 or more per cm².

22. A biochemical analyzing method comprising steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating radiation energy and specifically binding a substance derived from a living organism and labeled with the radioactive labeling substance with the specific binding substances, thereby selectively labeling the plurality of absorptive regions with the radioactive labeling substance, superposing the biochemical analysis unit on a stimulable phosphor sheet on which a stimulable phosphor layer is formed, thereby exposing the stimulable phosphor layer to the radioactive labeling substance selectively contained in the plurality of absorptive regions, irradiating the stimulable phosphor layer exposed to the radioactive labeling substance with a stimulating ray to excite stimulable phosphor contained in the stimulable phosphor layer, photoelectrically detecting stimulated emission released from the stimulable phosphor layer to produce biochemical analysis data, and effecting biochemical analysis based on the thus produced biochemical analysis data.

23. A biochemical analyzing method in accordance with claim 22 wherein the stimulable phosphor layer of the stimulable phosphor sheet includes a plurality of stimulable phosphor regions formed by charging stimulable phosphor into a plurality of holes formed in a support made of a material capable of attenuating radiation energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to the radioactive labeling substance selectively contained in the plurality of absorptive regions of the biochemical analysis unit.

24. A biochemical analyzing method in accordance with claim 22 wherein the material capable of attenuating radiation energy has a further capability to attenuate light energy and the biochemical analyzing method further comprises the steps of preparing the biochemical analysis unit by selectively labeling the plurality of absorptive regions with a fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data.

25. A biochemical analyzing method in accordance with claim 23 wherein the material capable of attenuating radiation energy has a further capability to attenuate light energy and which further comprises steps of preparing the biochemical analysis unit by selectively labeling the plurality of absorptive regions with a fluorescent substance, irradiating the biochemical analysis unit with a stimulating ray, thereby stimulating the fluorescent substance, photoelectrically detecting fluorescence emission released from the fluorescent substance to produce biochemical analysis data, and effecting biochemical analysis based on the thus produced biochemical analysis data.

26. A biochemical analyzing method in accordance with claim 22 wherein the material capable of attenuating radiation energy has a further capability to attenuate light energy and which further comprises steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data.

27. A biochemical analyzing method in accordance with claim 23 wherein the material capable of attenuating radiation energy has a further capability to attenuate light energy and which further comprises steps of preparing the biochemical analysis unit by selectively labeling the plurality of absorptive regions with a labeling substance which generates chemiluminescent emission when it contacts a chemiluminescent substrate in addition to the radioactive labeling substance, causing the biochemical analysis unit to come into contact with the chemiluminescent substrate, photoelectrically detecting chemiluminescent emission released from the labeling substance to produce biochemical analysis data, and effecting biochemical analysis based on the thus produced biochemical analysis data.

28. A biochemical analyzing method in accordance with claim 22 wherein the surface of the absorptive substrate covered with the material capable of attenuating radiation energy and the material capable of attenuating radiation energy has a property of reducing the energy of radiation to ⅕ or less when the radiation travels in the material by a distance equal to that between neighboring absorptive regions.

29. A biochemical analyzing method in accordance with claim 22 wherein the surface of the absorptive substrate is covered with metal, thereby forming a covering.

30. A biochemical analyzing method in accordance with claim 22 wherein the surface of the absorptive substrate is covered with metal or alloy selected from a group consisting of gold, silver, copper, zinc, aluminum, titanium, tantalum, chromium, iron, nickel, cobalt, lead and tin and alloys thereof, thereby forming a covering.

31. A biochemical analyzing method in accordance with claim 22 wherein the absorptive substrate is formed of a porous material or a fiber material.

32. A biochemical analyzing method in accordance with claim 22 wherein the covering of the material capable of attenuating radiation energy has a thickness of 0.5 to 100 times of the maximum breadth of the individual absorptive regions.

33. A biochemical analyzing method in accordance with claim 32 wherein the covering of the material capable of attenuating radiation energy has a thickness of 1 to 10 times the maximum breadth of the individual absorptive regions.

34. A biochemical analyzing method in accordance with claim 22 wherein the biochemical analysis unit is formed with 10 or more absorptive regions.

35. A biochemical analyzing method in accordance with claim 22 wherein each of the plurality of absorptive regions formed in the biochemical analysis unit has a size of less than 5 mm².

36. A biochemical analyzing method in accordance with claim 22 wherein the plurality of absorptive regions are formed in the biochemical analysis unit at a density of 10 or more per cm².

37. A biochemical analyzing method comprising steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and specifically binding a substance derived from a living organism and labeled with a fluorescent substance with the specific binding substances, thereby selectively labeling the plurality of absorptive regions with the fluorescent substance, irradiating the biochemical analysis unit with a stimulating ray, thereby exciting the fluorescent substance selectively contained in the plurality of the absorptive regions of the biochemical analysis unit, photoelectrically detecting fluorescence emission released from the fluorescent substance selectively contained in the plurality of the absorptive regions of the biochemical analysis unit to produce biochemical analysis data, and effecting biochemical analysis based on the thus produced biochemical analysis data.

38. A biochemical analyzing method in accordance with claim 37 wherein the surface of the absorptive substrate covered with the material capable of attenuating light energy and the material capable of attenuating light energy has a property of reducing the energy of light to ⅕ or less when the light travels in the material by a distance equal to that between neighboring absorptive regions.

39. A biochemical analyzing method in accordance with claim 37 wherein the surface of the absorptive substrate is covered with metal, thereby forming a covering.

40. A biochemical analyzing method in accordance with claim 37 wherein the absorptive substrate is formed of a porous material or a fiber material.

41. A biochemical analyzing method in accordance with claim 37 wherein the covering of the material capable of attenuating radiation energy has a thickness of 0.5 to 100 times of the maximum breadth of the individual absorptive regions.

42. A biochemical analyzing method in accordance with claim 37 wherein the covering of the material capable of attenuating radiation energy has a thickness of 1 to 10 times the maximum breadth of the individual absorptive regions.

43. A biochemical analyzing method in accordance with claim 37 wherein the biochemical analysis unit is formed with 10 or more absorptive regions.

44. A biochemical analyzing method in accordance with claim 37 wherein each of the plurality of absorptive regions formed in the biochemical analysis unit has a size of less than 5 mm².

45. A biochemical analyzing method in accordance with claim 37 wherein the plurality of absorptive regions are formed in the biochemical analysis unit at a density of 10 or more per cm².

46. A biochemical analyzing method comprising steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and specifically binding a substance derived from a living organism and labeled with a labeling substance which generates chemiluminescent emission when it contacts a chemiluminescent substrate with the specific binding substances, thereby selectively labeling the plurality of absorptive regions with the labeling substance which generates chemiluminescent emission when it contacts a chemiluminescent substrate, causing the biochemical analysis unit to come into contact with the chemiluminescent substrate, photoelectrically detecting chemiluminescent emission released from the labeling substance selectively contained in the plurality of the absorptive regions of the biochemical analysis unit to produce biochemical analysis data, and effecting biochemical analysis based on the thus produced biochemical analysis data.

47. A biochemical analyzing method in accordance with claim 46 wherein the surface of the absorptive substrate covered with the material capable of attenuating light energy and the material capable of attenuating light energy has a property of reducing the energy of light to ⅕ or less when the light travels in the material by a distance equal to that between neighboring absorptive regions.

48. A biochemical analyzing method in accordance with claim 46 wherein the surface of the absorptive substrate is covered with metal, thereby forming a covering.

49. A biochemical analyzing method in accordance with claim 46 wherein the absorptive substrate is formed of a porous material or a fiber material.

50. A biochemical analyzing method in accordance with claim 46 wherein the covering of the material capable of attenuating radiation energy has a thickness of 0.5 to 100 times of the maximum breadth of the individual absorptive regions.

51. A biochemical analyzing method in accordance with claim 46 wherein the covering of the material capable of attenuating radiation energy has a thickness of 1 to 10 times the maximum breadth of the individual absorptive regions.

52. A biochemical analyzing method in accordance with claim 46 wherein the biochemical analysis unit is formed with 10 or more absorptive regions.

53. A biochemical analyzing method in accordance with claim 46 wherein each of the plurality of absorptive regions formed in the biochemical analysis unit has a size of less than 5 mm².

54. A biochemical analyzing method in accordance with claim 46 wherein the plurality of absorptive regions are formed in the biochemical analysis unit at a density of 10 or more per cm².

55. A biochemical analyzing method comprising steps of preparing a biochemical analysis unit by spotting specific binding substances whose sequence, base length, composition and the like are known in a plurality of absorptive regions formed spaced apart from each other by covering a surface of an absorptive substrate made of an absorptive material with a material capable of attenuating light energy and specifically binding a substance derived from a living organism and labeled with a labeling substance which generates chemiluminescent emission when it contacts a chemiluminescent substrate with the specific binding substances, thereby selectively labeling the plurality of absorptive regions with the labeling substance which generates chemiluminescent emission when it contacts a chemiluminescent substrate, causing the plurality of absorptive regions of the biochemical analysis unit to come into contact with the chemiluminescent substrate, thereby causing the plurality of absorptive regions to release chemiluminescent emission, superposing the biochemical analysis unit whose plurality of the absorptive regions are releasing chemiluminescent emission and a stimulable phosphor sheet formed with a stimulable phosphor layer, exposing the stimulable phosphor layer to chemiluminescent emission released from the plurality of absorptive regions of the biochemical analysis unit, thereby storing an energy of chemiluminescent emission in the stimulable phosphor layer of the stimulable phosphor sheet, irradiating the plurality of absorptive regions of the biochemical analysis unit with a stimulating ray, thereby exciting stimulable phosphor contained in the stimulable phosphor layer, photoelectrically detecting stimulated emission released from the stimulable phosphor layer of the stimulable phosphor sheet to produce biochemical analysis data, and effecting biochemical analysis based on the thus produced biochemical analysis data.

56. A biochemical analyzing method in accordance with claim 30 wherein the stimulable phosphor layer of the stimulable phosphor sheet includes a plurality of stimulable phosphor regions formed by charging stimulable phosphor into a plurality of holes formed in a support made of a material capable of attenuating light energy in accordance with the same pattern as that of the plurality of absorptive regions formed in the absorptive substrate and the stimulable phosphor layer is superposed on the biochemical analysis unit so that the plurality of stimulable phosphor regions face the plurality of absorptive regions formed in the absorptive substrate, thereby exposing the plurality of stimulable phosphor regions to chemiluminescent emission released from the plurality of absorptive regions of the biochemical analysis unit.

57. A biochemical analyzing method in accordance with claim 55 wherein the surface of the absorptive substrate covered with the material capable of attenuating light energy and the material capable of attenuating light energy has a property of reducing the energy of light to ⅕ or less when the light travels in the material by a distance equal to that between neighboring absorptive regions.

58. A biochemical analyzing method in accordance with claim 55 wherein the surface of the absorptive substrate is covered with metal, thereby forming a covering.

59. A biochemical analyzing method in accordance with claim 55 wherein the absorptive substrate is formed of a porous material or a fiber material.

60. A biochemical analyzing method in accordance with claim 55 wherein the covering of the material capable of attenuating radiation energy has a thickness of 0.5 to 100 times of the maximum breadth of the individual absorptive regions.

61. A biochemical analyzing method in accordance with claim 55 wherein the covering of the material capable of attenuating radiation energy has a thickness of 1 to 10 times the maximum breadth of the individual absorptive regions.

62. A biochemical analyzing method in accordance with claim 55 wherein the biochemical analysis unit is formed with 10 or more absorptive regions.

63. A biochemical analyzing method in accordance with claim 55 wherein each of the plurality of absorptive regions formed in the biochemical analysis unit has a size of less than 5 mm².

64. A biochemical analyzing method in accordance with claim 55 wherein the plurality of absorptive regions are formed in the biochemical analysis unit at a density of 10 or more per cm².
